(19) United States
(12) Patent Application Publication
(10) Pub. No.: US 2016/0113567 A1
(43) Pub. Date: Apr. 28, 2016

(54) SYSTEMS AND METHODS FOR DIAGNOSIS OF DEPRESSION AND OTHER MEDICAL CONDITIONS

(71) Applicants: Laszlo OSVATH, Dundas (CA); Colin SHAPIRO, Dundas (CA)

(72) Inventors: Laszlo Osvath, Dundas (CA); Colin Shapiro, Dundas (CA)

(21) Appl. No.: 14/894,890

(22) PCT Filed: May 27, 2014
(86) PCT No.: PCT/CA2014/000460

§ 371 Date: Nov. 30, 2015

Related U.S. Application Data
(60) Provisional application No. 61/828,162, filed on May 28, 2013.

(51) Int. Cl. A61B 5/00; A61B 5/08; A61B 5/16; A61B 5/0478; A61B 5/0476; A61B 5/04

(52) U.S. Cl. CPC A61B 5/4812; A61B 5/0476; A61B 5/04012; A61B 5/165; A61B 5/0478; A61B 5/4088; A61B 5/08

(57) ABSTRACT

According to some aspects, one or more systems and methods for the diagnosis of a medical condition, such as depression, based on an analysis of sleep information. In some embodiments, the diagnostic system includes at least one recorder for recording sleep information about a patient, and at least one analyzer adapted to analyze the sleep information and determine whether the patient is experiencing the medical condition.

### RELATED APPLICATIONS

This application claims the benefit of U.S. Provisional Patent Application Ser. No. 61/828,162 filed May 28, 2013, the entire contents of which are hereby incorporated by reference herein.

### TECHNICAL FIELD

The embodiments described herein relate to systems and methods for diagnosing depression, and in particular to systems and methods for diagnosis of depression based on analysis of sleep information.

### INTRODUCTION

Human emotional states can generally be divided into two categories (called mood and affective states) based on the persistence of each state. Mood is generally considered to be a sustained emotional state that lasts for a few weeks or more. On the other hand, affective state (or affect) generally refers to a brief emotional response that is normally transitory in nature.

In general, affective responses are supposed to reinforce behaviors and serve important biological functions in mammalian physiology. However, some of these affective responses, such as euphoria, depression and anxiety, can become disturbed, persistent and dominant. When this happens, they can be characterized as an illness or medical condition, and may require treatment.

Depression is a particularly problematic medical condition, and is one of the most debilitating, costly, and stigmatized illnesses of our times. It is believed to affect an estimated 350 million people in communities all over the world, and on average about 1 in 20 people have reported having an episode of depression within the past year.

Unfortunately, notwithstanding the seriousness of depression, the current techniques for its diagnosing and guiding treatment are generally inadequate. For example, depression may be diagnosed by reviewing the clinical symptoms of a patient, such as by using the criteria contained in the Diagnostic and Statistical Manual of Mental Disorders (DSM-IV). DSM-IV is designed to identify a mood disorder such as depression by examining three elements: mood episodes, descriptors of most recent episode, and recurrence descriptors.

However, the DSM-IV techniques are problematic, particularly since examining these three elements requires input from the patients, including their ability to recognize and describe their own feelings. This ability can vary from patient to patient, especially for different cultural backgrounds, and tends to create inconsistencies in the results. Moreover, symptoms of depression can vary greatly between different patients. As a result the DSM-IV method for diagnosing depression tends to be subject to systematic error and often results in false results.

There are some physiological tests that attempt to help diagnose depression. Among these physiological tests are the dexamethasone suppression test, the tyrotropin releasing hormone stimulation test, the growth hormone response to insulin-induced hypoglycemia test, and the plasma cortisol level test. Unfortunately these physiological tests tend to be inconsistent and may be unreliable when used for diagnosis.

In some cases, it may be possible to diagnose depression by conducting a psychiatric interview of a patient. However, this approach tends to be heavily dependent on the abilities of the interviewer(s) and other factors that make it subjective and somewhat unreliable.

### BRIEF DESCRIPTION OF THE DRAWINGS

Some embodiments will now be described, by way of example only, with reference to the following drawings, in which:

FIG. 1 is a schematic diagram illustrating a system for diagnosing depression according to one embodiment;

FIG. 2 is a schematic diagram of a graphical user interface for a diagnosis system according to one embodiment;

FIG. 3 is a schematic diagram of functional components of a diagnosis system according to one embodiment;

FIG. 4 is a detailed diagram of an analyzer module of a diagnosis system according to one embodiment;

FIG. 5 is an diagram showing an example of sleep staging and a corresponding digital period analysis (DPA) for two random samples according to one embodiment;

FIG. 6 is an diagram showing an exemplary estimate of REM density according to one embodiment;

FIG. 7 is a schematic diagram of functional components of a REM density estimator according to one embodiment;

FIG. 7a is a diagram of an example of REM activity on EOG channels;

FIG. 8 is graph comparing beta bilateral coherency for adults between a normal individual and a depressed individual;

FIG. 9 is graph comparing beta delta coherency in the left hemisphere for adults between a normal individual and a depressed individual;

FIG. 10 is graph comparing beta delta coherency in the right hemisphere for adults between a normal individual and a depressed individual;

FIG. 11 is graph comparing theta bilateral coherency (TCOH) in adults between a normal individual and a depressed individual;

FIG. 12 is graph comparing beta delta coherency in the right hemisphere for children between a normal individual and a depressed individual;

FIG. 13 is graph comparing beta delta coherency in the left hemisphere for children between a normal individual and a depressed individual;

FIG. 14 is an exemplary drawings of a model artificial neuron according to one embodiment;

FIG. 15 is an exemplary drawing of an artificial neural network according to one embodiment;

FIG. 16 is an exemplary drawing of an artificial neural network according to another embodiment; and

FIG. 17 is an exemplary graph of an estimate of coherence according to one embodiment.

### DESCRIPTION OF SOME PARTICULAR EMBODIMENTS

For simplicity and clarity of illustration, where considered appropriate, reference numerals may be repeated among the figures to indicate corresponding or analogous elements or steps. In addition, numerous specific details are set forth in order to provide a thorough understanding of the exemplary embodiments described herein. However, it will be understood by those of ordinary skill in the art that the embodiments described herein may be practiced without these specific details. In other instances, well-known methods, procedures and components have not been described in detail so as not to obscure the embodiments generally described herein.

Furthermore, this description is not to be considered as limiting the scope of the embodiments described herein in any way, but rather as merely describing the implementation of various embodiments.

In some cases, the embodiments of the systems and methods described herein may be implemented in hardware, in software, or a combination of hardware and software. For example, some embodiments may be implemented in one or more computer programs executing on one or more programmable computing devices that include at least one processor, a data storage device (including in some cases volatile and non-volatile memory and/or data storage elements), at least one input device, and at least one output device.

In some embodiments, a program may be implemented in a high level procedural or object-oriented programming and/or scripting language to communicate with a computer system. However, the programs can be implemented in assembly or machine language, if desired. In any case, the language may be a compiled or interpreted language.

In some embodiments, the systems and methods as described herein may also be implemented as a non-transitory computer-readable storage medium configured with a computer program, wherein the storage medium so configured causes a computer to operate in a specific and predefined manner to perform at least some of the functions as described herein.

As briefly described above, known methods for diagnosing depression tend to be inadequate. In particular, existing diagnosis methods tend to be laborious, costly, subjective, time consuming, incomplete (i.e., they may not cover the full spectrum of the illness), or some combination thereof. Moreover, some known methods for diagnosing depression may be available only through highly trained medical personnel (i.e., a psychiatrist), may not be easily reproducible, and may be subject to error or very difficult to standardize.

At least some of the teachings herein are directed at systems and methods for diagnosing depression which may provide for improved results as compared to at least some previous known techniques.

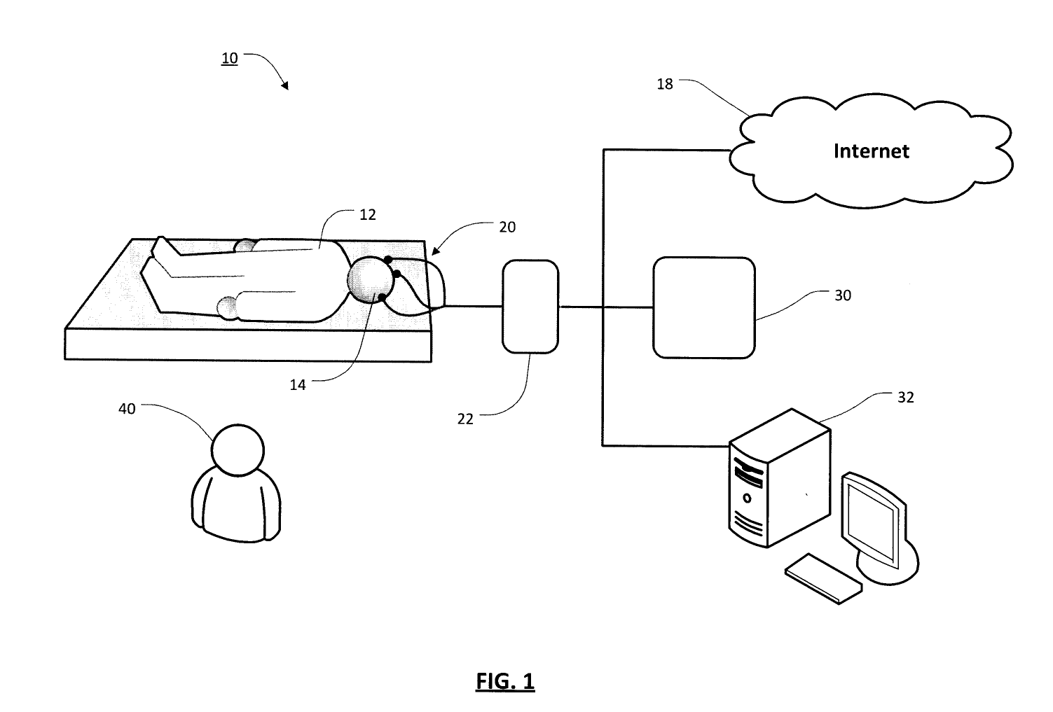

Turning now to FIG. 1, illustrated therein is a schematic diagram of a system 10 for diagnosing depression according to one embodiment.

In general, the system 10 may be operable for use in various locations, such as a sleep clinic or laboratory, or other medical facility. In some embodiments, the system 10 may be operable in another environment, such as in a person's home.

Generally, the system 10 uses electroencephalography (EEG) to monitor the sleep patterns of a patient (i.e. the patient 12 in FIG. 1). Electroencephalography (EEG) refers to recording measurements of electrical activity along a patient's scalp. More particularly, an EEG measures voltage fluctuations that result from changing current flows within the neurons of the patient's brain.

An EEG can be useful for monitoring a patient's sleep patterns, since brain function varies during waking and the different stages of sleep. This variation can be detected by the EEG. In particular, as a person sleeps their brain generally switches between different stages of activity, with different brain wave patterns associated with each stage.

For example, stage 1 is the beginning of a sleep cycle, which is relatively light sleep. During this stage, the brain produces alpha waves. During stage 2 sleep, the brain tends to produce theta waves, and can produce rapid, rhythmic brain wave activity known as sleep spindles. In stage 3, which is a transitional stage between light and deep sleep, the brain begins to produce delta waves, which are deep and slow. In stage 4, the brain is in a deep sleep and produces many deep and slow delta waves. Depending on the particular sleep classification system being used, in some case stage 3 and stage 4 sleep may be grouped together and referred to simply as slow-wave sleep (SWS).

Finally, in stage 5, the brain enters Rapid Eye Movement (REM) sleep, also known as active sleep. This is the stage in which the majority of dreaming will occur.

As shown in FIG. 1, to monitor the patient's 12 sleep patterns, electrodes 20 of an electroencephalograph 22 (the EEG measuring device) may be coupled to the scalp 14 of the patient 12 to observe brain wave activity.

In some embodiments, the electrodes 20 could be placed onto the scalp 14 using a conductive gel or paste. This technique may be particularly suitable where the system 10 is being used at a sleep clinic or other medical facility, and where another person 40 (i.e., a sleep clinician) may be available to assist with properly placing the electrodes on the scalp 14.

In some embodiments, the electrodes 20 could be located within a cap or net that can then be placed on the head of the patient 12 so that the electrodes 20 are properly positioned on the scalp 14. This approach may be particularly suitable where the system 10 is being used at a person's home or other similar environment, since it may allow the placement of the electrodes 20 on the scalp 14 to be controlled more easily, especially when a clinician may not be available to assist with electrode placement.

In general, brainwave information that is received via the electrodes 20 may be processed by the electroencephalograph 22 to generate some sleep data that is representative of the sleeping behavior of the patient 12. Depending on the particular configuration of the system 10, this sleep data may then be sent to one or more devices or diagnostic tools for analysis. In some cases, the sleep data may be in a raw state (i.e., generally unprocessed brainwave data). In other cases, the sleep data may be processed (i.e., converted to a hypnogram or other processed data).

In some embodiments, the sleep data from the electroencephalograph 22 may be sent to a diagnosis device 30. The diagnosis device 30 may for instance be a stand-alone device that is operable to interpret the sleep data and generate a depression diagnosis for the patient 12.

In some cases, this diagnosis may be done by the diagnosis device 30 without any intervention by a clinician or other user. In other cases, the diagnosis device 30 may receive input from a user, for example to help calibrate the diagnosis (i.e., to compensate for certain variables such as gender, age, and so on).

In some cases, the diagnosis device 30 may have dedicated hardware components or software modules (or both), and may have various form factors. For instance, in some embodiments, the diagnosis device 30 may be a portable electronic device that may include a display screen, an input device, a power source, and other functional components. This embodiment may be particularly useful where the diagnosis device 30 is adapted to be used in a home environment.

In some cases, the diagnosis device 30 and electroencephalograph 22 may be provided as part of the same physical unit. For instance, the diagnosis device 30 and electroencephalograph 22 may have integrated hardware or software components (or both) that are provided within a single unitary housing or body.

In other embodiments, the diagnosis device 30 and EEG measuring device 20 may be separate and distinct, and may communicate in various ways, such as by a wired or wireless communication channel.

In some embodiments, sleep data from the electroencephalograph 22 may be sent to a processing device 32 that is operable run a diagnostic software application for diagnosing depression. In general, the processing device 32 may be any suitable computing device, such as a server, personal computer, laptop, tablet, smartphone, etc. In particular, the processing device 32 may be a general purposes computer running a software application that is designed to interpret the sleep data and generate a diagnosis for the patient 12 therefrom according to the teachings herein.

In general, the processing device 32 may include one or more processors, one or more data storage devices, one or more input and output devices, and so on as will be suitable for controlling the operation of the software application.

In some embodiments, the sleep data from the electroencephalograph 22 may be sent for analysis to a different location. For example, the sleep data may be sent over the internet 18 or another communications network to a diagnosis system that is remotely located from the patient 12. This approach may be particularly suitable where the patient 12 is undergoing the EEG analysis at home, as it may allow diagnosis to be provided as a service without requiring a diagnostic device to be physically present with the patient 12 and/or the electroencephalograph 22.

In some embodiments, as briefly discussed above, the sleep data from the EEG measuring device 20 may be raw sleep data, such as measured electrical activity related to the brainwaves of the patient 12.

In other embodiments, the sleep data from the EEG measuring device 22 may be processed to generate processed data (which might include a hypnogram, for example) that is then sent to the diagnostic device 30, the processing devise 32, and so on, so that the patient 12 can be diagnosed.

In some cases, raw sleep data can be automatically processed to generate the processed sleep data, for example by a hardware or software application designed to interpret EEG data and generate a hypnogram (or other processed data) therefrom that shows various stages of sleep as a function of time.

In other embodiments, the raw sleep data can be manually processed (i.e., by the clinician 40 or other user) who may be trained to interpret raw EEG data and generate a hypnogram or other processed data.

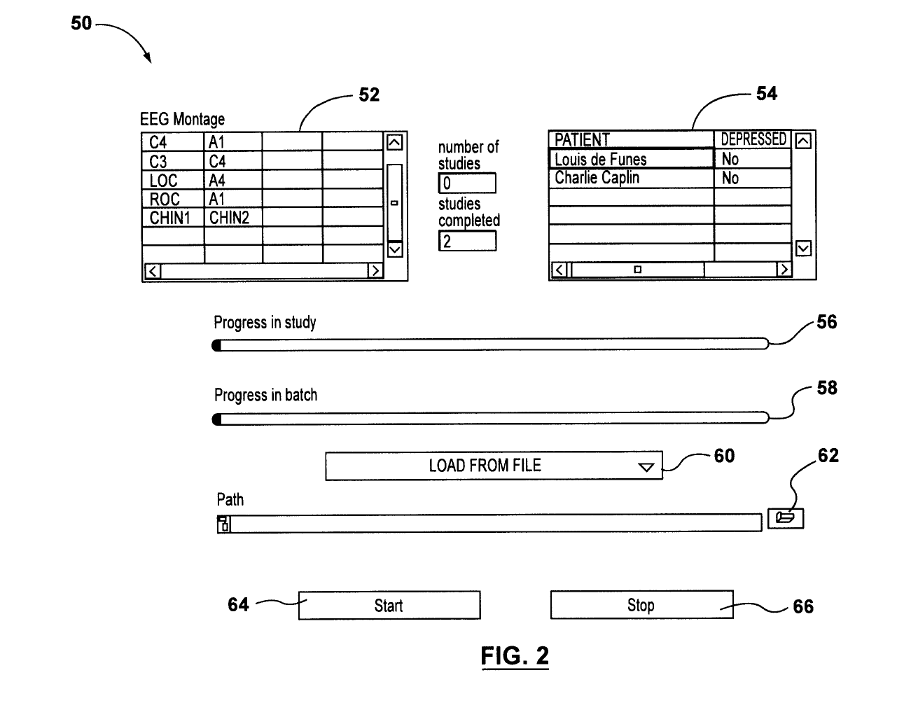

Turning now to FIG. 2, a schematic diagram of a graphical user interface (GUI) 50 for a diagnosis system is shown according to one embodiment. For example, the GUI 50 may be presented on the diagnostic device 30, on the processing device 32, as a web service (i.e., as a webpage available over the internet 18), or in some other context.

In general, the GUI 50 may contain various controls and display information that allow a user to perform a diagnosis on one or more patients. For example, the GUI 50 may contain a first display area 52 that shows information about an EEG montage, and a second display area 54 that contains the results of depression diagnosis for one or more patients.

The GUI 50 may also contain one or more progress indicators (i.e., progress bars 56, 58) that are indicative of the progress of one more aspects of the diagnosis, such as the analysis for a particular patient, the analysis of a group of patients, and so on.

The GUI 50 may also include controls for controlling the diagnosis. For example, one or more controls may allow a user to select a mode of operation and load information from a particular file (i.e., a file that contains sleep data, such as raw sleep data or processed sleep data). In this embodiment, the controls include a drop down list mode control 60 and a file open control 62.

Finally, the GUI 50 may also include other controls, such as buttons 64, 66, that are operable for starting and stopping the diagnosis.

During use, a user may pick an input folder or file that contains sleep data (i.e., using the file open control 62), and select a mode of operation for the diagnosis system from one or more particular modes (i.e., using the mode control 60). In this embodiment, some of the modes include “Diagnose”, “Load Data from Files”, “Train” and “Cross-Validation Test”.

The Diagnose mode of operation may be the most commonly used, and allows the GUI 50 to initiate diagnosis of a particular patient or patients based on sleep data that is loaded into the appropriate folder.

The Train mode may allow a user to create a different training set that can be used for diagnosis, instead of various pre-computed diagnostic templates that may have already been prepared for the diagnostic system.

The Cross-Validation Test may allow proper operation of the diagnosis system to be checked, for example by running the diagnosis system against a known reference set (i.e., a pre-computed or user created reference set).

In this embodiment, the Load Data From Files is an auxiliary mode that may be useful for adjusting the reference data set. In particular, it may allow synthetic data sets to be reused, and which are created prior to computing diagnostic parameters, thus allowing a synthetic data generation process to be bypassed.

When the Diagnose mode is engaged (i.e., by activating the start button 64), the diagnosis system will look for any patient files in an appropriate input folder. If patient files are located, the diagnosis system can start loading data associated with these patients and begin its analysis. Current progress may be indicated by the progress bars 56, 58, which in this embodiment can show progress both for the current patient being analyzed, as well as the overall progress for a number of different patients.

As patients are analyzed, the second display area 54 can be updated with results. For example, in one embodiment, the result for each patient might be displayed from the list of NO (meaning that the patient is not depressed), YES (meaning that the patient is depressed), NOT TESTED (for example if for some reason the patient was not able to be tested), or UNKNOWN (if the diagnosis system cannot reach a definitive conclusion).

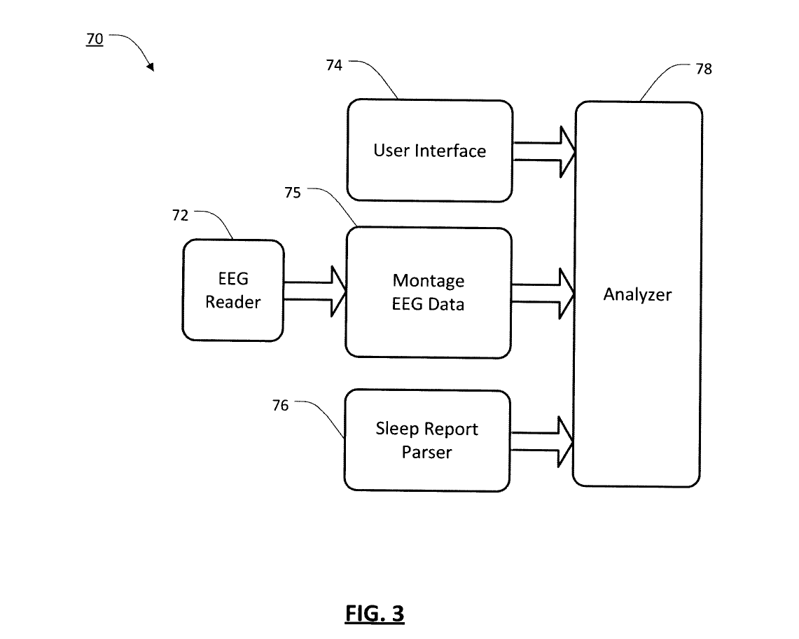

Turning now to FIG. 3, illustrated therein is a schematic diagram of functional components of a diagnosis system 70 according to one embodiment. In general, these functional components could be executed in hardware, software, or some combination thereof.

In general, the diagnosis system 70 includes an EEG reader 72 that is operable to read sleep data files (i.e., the raw data files). In some cases, the EEG reader may decompress sleep data received from an electroencephalograph (i.e., electroencephalograph 22) and then send this data to a montaging block 75.

The montaging block 75 is operable to prepare the sleep data for further analysis by an analyzer 78 as will be described in further detail below.

In some embodiments, a user interface 74 may be used to control one or more aspects of the diagnosis system 70. For example, the user interface 74 may be the GUI 50 described above or some other suitable user interface.

In some embodiments, the diagnosis system 70 may include a sleep report parser 76. When appropriate, the sleep report parser 76 may load and extract relevant data from previously prepared sleep reports (i.e., existing sleep reports for the patient 12), if such sleep reports exist and are available. These existing sleep reports may be analyzed and may in some cases be helpful for determining whether the patient has any biological markers that are associated with depression.

It should be noted that the use of existing sleep reports is not required, and in some cases may be undesirable. In particular, prior sleep reports may have been prepared in different sleep clinics or laboratories, and variations in how each particular clinic prepares its sleep reports may impact the consistency between prior sleep reports, potentially limiting their usefulness.

Thus, in some cases, the diagnosis system 70 may be operable without including any data from prior sleep reports, even when prior sleep reports are available. This may be done to avoid possible inter-laboratory variation in the sleep reports.

In some cases, the diagnosis system 70 may be used without receiving EEG data via the EEG reader 72, in which case the sleep report parser 76 would be used to send only prior sleep reports to the analyzer 78. This approach may be appropriate when a particular user wants to use his or her own sleep staging and scoring, without generating any new sleep data. For example, a sleep clinic may have already performed a number of sleep studies of a particular patient, and may desire to use these existing sleep studies as the basis for a diagnosis.

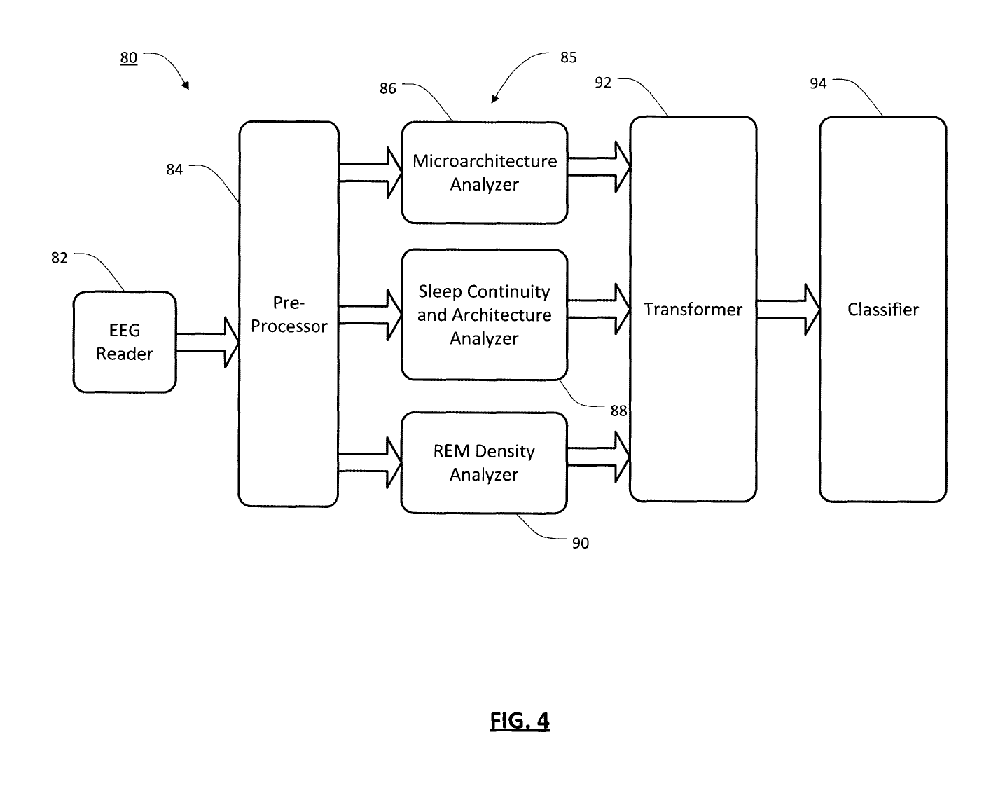

Turning now to FIG. 4, further details of an analyzer module for a diagnosis system 80 are shown according to one embodiment.

In this embodiment, the EEG reader 82 sends data to a pre-processor 84, which is operable to prepare the sleep data for analysis (i.e., by formatting the data as may be required for use by the analyzers and so on). The pre-processor 84 will then send this data to a montaging block 85 that includes one or more analyzers.

In this specific embodiment, the montaging block 85 includes three analyzers: a microarchitecture analyzer 86, a sleep continuity and architecture analyzer 88, and a REM density analyzer 90.

The various analyzer modules 86, 88, 90 of the montaging block 85 may create a set of time series that characterize particular information about the sleep behavior of the patient 12, such as the patient's EEG data, eye movements, and muscle tone levels during a particular sleep study.

These time series can then be sent to a transformer 92. The transformer 92 in turn can convert the time series in a vector of parameters. When properly tuned, the transformer 92 acts as an adapter between the different data analyzers (i.e., the microarchitecture analyzer 86, sleep continuity and architecture analyzer 88, and REM density analyzer) so that the data can be interpreted by a classifier 94 to render a diagnosis.

In general, the classifier 94 may be operable to build boundaries between normal and depressed patients in a multidimensional state space. Based on these boundaries, the classifier 94 can reach a binary decision about whether the patient is or not depressed (i.e. the classifier 94 may generate a YES or NO answer about whether the patient 12 is depressed).

In some embodiments, instead of a YES or NO the classifier 94 may provide some indication of the severity of the depression (i.e., MILD, MODERATE, SEVERE, etc.)

In some embodiments, the classifier 94 may provide other results (e.g., UNKNOWN etc.) where it is unable to reach a definite conclusion in regards to the depression of the patient 12.

In some embodiments, the decision boundaries of the classifier 94 are built from one or more training sets, and the patient that is being diagnosed (i.e., patient 12) is compared to pre-existing knowledge about normal populations to look for patterns associated with depression.

More specifically, it has been discovered that several sleep related characteristics are influenced by major depressive disorders (MDD). Individually, each of these sleep related characteristics may be inadequate as biological sleep markers of depression, since they may be subject to individual variability between patients and hence may not be wholly reliable for an accurate diagnosis.

However, by fusing a plurality of sleep related characteristics together, it is believed that a multidimensional descriptor of the state of the patient can be defined, and which may be generally useful for diagnosing depression in that patient. In particular, nonlinear classification methods may be able to reliably separate depressed and normal subjects based on analyzing a plurality biological markers.

### Characterizing Sleep

Several methods of classification that integrate various aspects of sleep are chronobilogical, microarchitectural, macroarchitectural, and continuity of sleep, as will be discussed further herein. These characteristics are modulated by the presence of major depressive disorder (MDD).

### Chronobiological Markers

The sleep and wake states in humans and other mammals tend to follow a cyclic pattern that is regulated by an internal circadian clock in the suprachiasmatic nucleus, a structure in the anterior hypothalamus. When humans are removed from external cues, they will maintain an endogenous periodicity of their circadian rhythm. In humans this period is slightly over 24 hours.

In addition to the 24 hour circadian rhythm, humans also experience a rhythm with a shorter period called an ultradian rhythm (also referred to as a sleep-wake cycle). One candidate biological marker for diagnosing depression is a phase shift of the ultradian rhythm, which in general is described by an early REM stage.

In order to study the frequency spectrum of a very slowly evolving phenomenon (like the ultradian rhythm), a sleep study for a particular patient should contain at least one period of the periodic behaviour.

Since, the normal ultradian rhythm has a period of about ninety minutes, a sleep record of at least 90 minutes long should be used. Indeed, many sleep records are several hours in length (in some cases up to 8 hours in length or more), which should provide sufficient time to review the variability in the ultradian rhythm.

### Continuity

The continuity of sleep may be measured in terms of the following parameters that can be extracted from polysomnographic (PSG) studies. These parameters include:

sleep latency (SL);

wake after sleep onset (WASO);

number of awakenings (NWAK);

sleep efficiency (SE);

and total sleep time (TST).

### Macroarchitecture

The macroarchitectural abnormalities in sleep may include the following parameters:

altered distribution of slow-wave sleep (i.e., patient lack the traditional attenuation pattern across the night);

reduced slow-wave sleep (in minutes and/or percent);

decreased latency to the first episode of REM sleep (i.e. reduced REM latency);

prolonged first REM period;

increased REM percent (if not REM time in minutes); and

increased REM density (i.e. eye movements per minute of REM sleep).

The altered distribution of sleep in depression was noted to have resemblance to alterations observed due to aging (with the exception of REM density, which is more or less invariable with age).

Conventional wisdom is that parameters like REM latency alone are unsuitable as sleep markers indicative of depression. Thus, considering architectural elements or continuity descriptors of sleep individual as potential sleep markers may be less promising than looking at the record as a whole. However, by reviewing the sleep record as a whole, it is presently believed that it may be possible to provide a diagnosis of depression.

### Microarchitecture

In addition to studying the diminution of delta wave amplitude and incidence and increase in amplitudes in the beta band, the study of the microarchitectuire of sleep employed a technique called digital period analysis (DPA) that allows for continuous measure of delta activity, as contrasted to the standard PSG technique where a specified proportion of an epoch (e.g., a 30 second epoch) has to be covered by delta activity, with variations being artificially left out.

The coherence of EEG activity in various spectral bands appears to provide significant results in discriminating between depressed persons and controls. Further microarchitectural variables that may be indicative of depression are whole night beta and gamma activity during NREM (non-REM sleep), and around sleep onset.

In one case, the degree of association between sleep disturbance and symptoms of depression were studied, and it was determined that sleep and depression may be strongly related phenomena.

Relevant depression symptoms were found to be the core symptoms of depression and not neurovegetative symptoms while on the sleep side the relevant parameters were found to be mostly NREM variables.

The clinical relevance of sleep continuity disturbance appears to be that people with persistent insomnia have higher probability of developing depression and those patients with no improvement of sleep continuity after antidepressant treatment have higher chances of relapse than those with improved sleep continuity.

The parameters related to the architecture of sleep are mainly REM latency, REM density and SWS time. Out of these parameters it appears that REM density may be correlated to severity of depression, particularly since REM latency can be a predictor of treatment outcome. More particularly, a reduced REM latency is associated with poor treatment outcomes.

### Coherence and Complex Coherency

The concepts of coherence and coherency will now be discussed. Coherence may be used in various fields for time delay estimation, as a measure of linear relationship between two processes, for system identification, and as a measurement of signal-to-noise (SNR) power ratio. To clarify the difference between coherence and coherency, the term “coherence” is the square of “coherency”.

In general, if a discrete stochastic process x is linearly related to a discrete stochastic process y, one can write:

G_{yy}(f)=|H(f)|²Gₓₓ(f)

In this equation, G_{yy} is the power spectrum of the process y, Gₓₓ is the power spectrum of process x, and H(f) is the transfer function. By definition, the cross power spectrum for this equation is:

G_{xy}=DFT(k_{xy})

where DFT is the discrete Fourier transformation operator, and k_{xy} is the covariance function between processes x and y.

Expanding the covariance and reversing the order of integration of the Fourier transform and expectation gives:

G_{xy}(f)=H(f)Gₓₓ(f)

Complex coherency is a function, defined as the ratio of the cross-power spectral density of two random processes, and the product of their auto-power spectral densities:

$${γ}_{xy} = \frac{{G}_{xy} ( f )}{\sqrt{{G}_{xx} ( f ) {G}_{yy} ( f )}}$$

The magnitude squared coherency, or “coherence”, is bounded and has support [0,1]:

C_{xy}=γ_{xy}²

In a linear relationship, by inserting the first two equations into the equation for coherency, one gets C_{xy}=1. As a first observation, it can be noted that the coherence can be interpreted as departure from a linear relationship in the case of two stationary random processes.

However, despite mentioning a linear relationship, this approach is not limited to linear processes. Any nonlinear process can be linearized to some extent, and the adequacy of such linearization can be evaluated. If a linear model is considered generally adequate (i.e., if it seems to be a reasonably good model), then the linear model can be used to provide valuable insight into the particular process being examined.

In the case of performing an identification task of a stationary process y, one can feed the process x into the input of a model, and then adjust the model by minimizing the least squares error between its output and the process y. This yields a frequency characteristic of the model:

$$H ( f ) = \frac{{G}_{xy}}{{G}_{xx}}$$

According to this equation, the frequency characteristic of the model is related to the squared coherence by:

$${C}_{xy} ( f ) = H ( f ) \sqrt{\frac{{G}_{yy}}{{G}_{yy}}}$$

The model in signal processing literature is called a filter and can be characterized by a set of coefficients that uniquely describe the model. This suggests that the coherence can be interpreted as an optimal (or at least desirable) normalized filter that minimizes (or at least greatly reduces) the error between the response of the filter to the process x and the process y. In a case of coherency, the model will describe the linear relationship between the two processes, process x and process y.

The error between the estimate and the modelled process is itself a random process. The power of the error process between y and its estimate is:

Gₑₑ=G_{yy}(f)[1−C_{xy}(f)]

This means that for large coherence the error power is small, whereas for small coherence the error power is large (depending on how much of the y process is explained by its estimator model).

The spectrum of a process can be considered as a sum of two terms, a desired part and an error part:

G_{yy}=G_{yy}C_{xy}G_{yy}(1−C_{xy})

The ratio of these components can be interpreted as either a linear-nonlinear power ratio, which is the fraction of power that is contained in the linear part of the relationship to the power contained in the nonlinear part of the relationship. The other interpretation is as a signal to noise ratio (SNR), which is a ratio of the desired part relative to the undesired (noise) part of a model:

$$\frac{{G}_{yy}}{{G}_{ee}} = \frac{{C}_{xy}}{1 - {C}_{xy}}$$

Complex coherency can be further interpreted using spectral representation theorem. According to this theorem a stochastic process can be represented by:

x(t)=∫_{-π}^{π}e^{iωt}dZₓ(ω),

where Zₓ is a another stochastic process, and for a given ω, Zₓ(ω) is a random variable. Describing each process as above, one then arrives at:

y(t)=∫_{-π}^{π}e^{iωt}dZ_{y}(ω),

Using this representation it can be shown that the complex coherency can be written:

$${C}_{xy} ( f ) = \frac{cov ( {dZ}_{x} ( f ) , {dZ}_{y} ( f ) )}{var ( {dZ}_{x} ( f ) ) var ( {dZ}_{y} ( f ) )}$$

From this equation, it can be observed that the complex coherency can be interpreted as the correlation coefficient for the random variables of the component processes Zᵢ of the two stochastic process x and y.

C_{xy} thus gives information on how x and y are linearly related. At a given frequency (f), C_{xy} measures the relationship between the random coefficients at a frequency f of two processes x and y.

### Digital Period Analysis

Digital period analysis (DPA) will now be discussed. Sleep studies often use the fractions of fixed time windows that include delta activity as an indication that a patient is in either stage 3 or stage 4 sleep. This is related to another form of signal analysis, called digital period analysis (DPA).

The frequency distribution of EEG waves is a multidimensional random process. To analyze an EEG, time can be discretized into units of 30 seconds called “epochs”. At a specific time (i.e., once in every 30 seconds), the EEG data will provide a stochastic distribution of frequencies, each representing a multidimensional random variable. (e.g., the distribution of delta waves at some time t is a one dimensional random variable, and the time evolution of a distribution of delta activity is a one dimensional random process).

Extending this principle to the multivariate case, and sectioning the stochastic process at time t, a momentary frequency distribution can be obtained. This distribution can then be partitioned into the sub-bands of the different brain waves of interest: delta (1-4 Hz), theta (4-6 Hz), and beta (16-32 Hz).

The multidimensional random process is a simplified model of sleep, similar to the relationship between an object and its shadow on a wall. The random process is expected to contain a strong ultradian component in concordance with the known ultradian variation of sleep, similar to the shadow preserving some resemblance to the original object.

It is generally possible to study the variation of each of the one dimensional random processes in isolation, in which case the interrelationships between various variables could be ignored.

On the other hand, a multivariate approach could be used that includes possible interactions between the processes. This multidimensional approach is believed to provide more meaningful results. In particular, including a number of interactions (in some cases, as many interactions as possible) may provide a more complete picture of sleep and better distinguish “normal” sleep from the sleep of a depressed person. These interactions can characterize the slipping of one ultradian, random component relative to some other one-dimensional ultradian random component of sleep.

A delay or advance of ultradian rhythms through modified REM latencies is believed to be useful for diagnosing depression. It is therefore helpful to determine if the degree of slipping of a one-dimensional random processes is coherent, or if it is accompanied by some dispersion, or frequency dependent slipping. In some cases, characterization of the dispersion of ultradian rhythms may also be a biological marker of depression.

In current sleep medicine practices, the analysis of sleep studies is usually performed in 30 seconds epochs. As part of standard methods of sleep staging, some stages of sleep are identified by using proportions of waves of a specified duration and amplitude. Instead of using continuous proportions, a fixed threshold may be applied; a particular epoch may be either sub-threshold or above this threshold and consequently called stage 3 or 4 accordingly.

The proportions of specific types of waves are informative of the characteristics of sleep. Using proportions can be considered a more accurate alternative for characterizing sleep as opposed to methods of power spectral analysis.

In particular, due to the fact that power spectral analysis is an averaging method, and due to the loss of phase information, the power spectrum (unlike the Fourier transform) does not preserve a one-to-one relationship to the original signal. As a consequence, the original signal cannot be restored from the power spectrum, and there can be different waves that have the same power spectrum.

In some cases, it would be helpful to have an accurate measure of the proportion of waves of different durations, as in a rolling distribution of waves in various frequency bands. To this end, a method of counting waves tends to be more suitable than the averaging method of power spectral analysis because of the closer relationship between spectral content and the original time-series.

According to some of the teachings herein, a specific wave has a duration and a corresponding frequency. Each specific wave is considered either to be in one band or another, and the sum of the duration of the waves is equal to the duration of the original time-series. This method is generally called Digital Period Analysis (DPA).

A variation on Digital Period Analysis (DPA) will now be described, where variations exist based on the filtering applied prior to segmentation and the segmentation method, with the goal of identifying possible wave boundaries.

In one example, samples of random processes were filtered with a digital band-pass Infinite Impulse Response (IIR) filter with −100 db/dec and pass-band (0.5 Hz, 70 Hz). A digital band-stop filter was also used for the line frequency. The band stop filter was created using a High-Pass filter with transition band (0.1, 0.5 Hz) with −100 db/dec and a Low-Pass filter with transition-band (70, 80 Hz) −100 db/dec.

The filtering operation transformed the data in a zero mean random variable. Original data is denoted on the two channels of interest x₁ and x₂ respectively. Each channel had a four dimensional sample of the random process. A section through the process at discrete time n, will be represented by the random vector:

x=[n_{δ}n_{θ}n_{β}]

The significance of the random components will become clear as the computation is undertaken. The computation of nᵢ where iε{δ,θ,β} proceeds as follows. First, define the operator that finds the zero crossings of a time series:

zₓ=Zero(x)={n|x[n−1]*x[n]≦0}

where x is a random variable. Then define the derivative operator D:

Dx=x[n]−x[n−1]

Using the operators D and Z, build the following random processes:

$${n}_{δ} = {\sum }_{i} ( zx [ i ] - zx [ i - 1 ] \geq \frac{{f}_{s}}{4} ) ( zx [ i ] - zx [ i - 1 ] \geq \frac{{f}_{s}}{4} ) ( zx [ i ] - zx [ i - 1 ] \leq {f}_{s} ) \frac{zx [ i ] - zx [ i - 1 ]}{fs}$$

which represents counting the waves that have a frequency in the delta range (i.e., 1-4 Hz). One can then build the set:

zdₓ=Zero(Dx),

and define the following two random processes:

$${n}_{θ} = {\sum }_{i} ( {zd}_{x} [ i ] - {zd}_{x} [ i - 1 ] \geq \frac{{f}_{s}}{7} ) ( {zd}_{x} [ i ] - {zd}_{x} [ i - 1 ] < \frac{{f}_{s}}{4} ) \frac{{zd}_{x} [ i ] - {zd}_{x} [ i - 1 ]}{fs}$$

$${n}_{β} = {\sum }_{i} ( {zd}_{x} [ i ] - {zd}_{x} [ i - 1 ] \geq \frac{{f}_{s}}{32} ) ( {zd}_{x} [ i ] - {zd}_{x} [ i - 1 ] < \frac{{f}_{s}}{16} ) \frac{{zd}_{x} [ i ] - {zd}_{x} [ i - 1 ]}{fs}$$

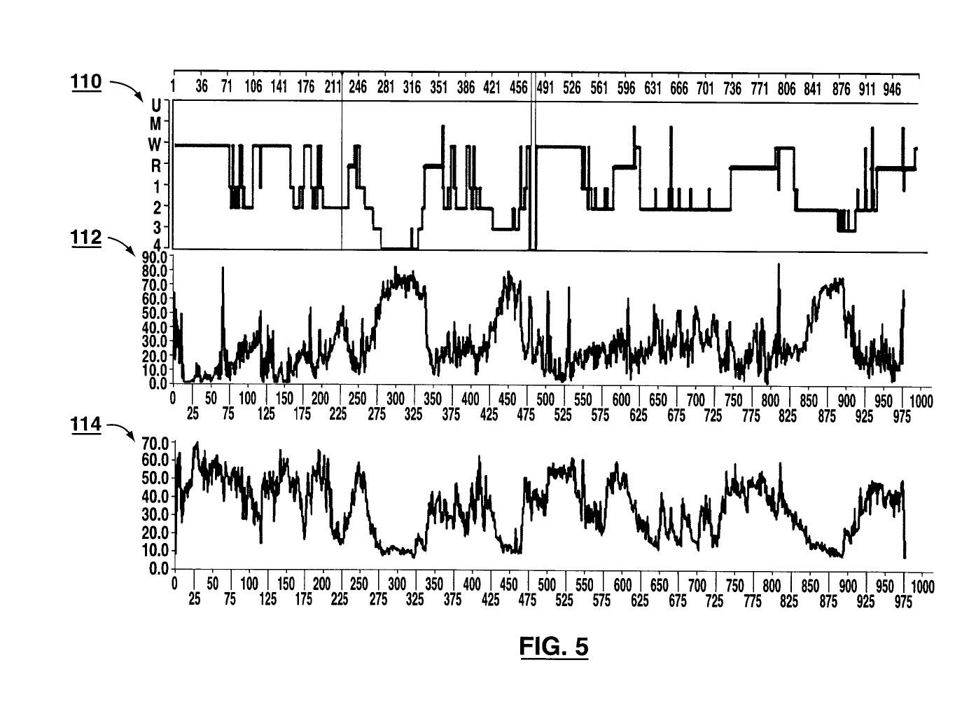

An exemplary illustration of sleep staging 110 and samples of the n_{δ} and n_{β} processes is presented in FIG. 5, namely n_{δ} (shown as the middle graph 112) and n_{β} (shown as the lower graph 114). The ordinate represents the percentage of an epoch covered with waves from the corresponding random process.

In order to compute estimates of coherence, estimates of auto spectra and cross spectra can be computed. For instance, one method is to use an overlapped fast Fourier transform. However, due to resolution in the range of about 18.5 mHz, long samples are generally needed and this method is not particularly suitable due to the limitations given by the sleep record duration. Another method amenable to short samples is the smoothed periodogram method:

G_{xy}(θ)=∫_{-π}^{π}N⁻¹|X(θ−λ)|²W(λ)dλ

where W is odd-length symmetric window, N is the width of the window, and X is the power spectral density of the process x. This equation is easier to compute in time domain:

$${G}_{xx} = {\sum }_{- M}^{M} {k}_{xx} [ n ] w [ n ] {e}^{- iθ n} with$$

$${k}_{xx} [ m ] = \frac{1}{N} {\sum }_{0}^{N - 1 - \vert n \vert} x [ i ] x [ i + \vert n \vert ]$$

A further simplification arises due to the relation between convolution and cross-covariance:

k_{xy}=x*[−n]*y[n] and similarly

kₓₓ=x*[−n]*x[n]

where, x* is the complex conjugate of x. Combining these equations, one gets the computational relations:

Gₓₓ(θ)=|DFT((x*[−n]*x[n])w[n])|

G_{xy}(θ)=|DFT((x*[−n]*y[n])w[n])|

These can then be used to get the computational relation for C_{xy}.

$${C}_{xy} = \frac{\vert FFT ( ( {x}^{*} [ - n ] * y [ n ] ) w [ n ] ) \vert \vert FFT ( ( {x}^{*} [ - n ] * y [ n ] ) w [ n ] ) \vert}{\vert FFT ( ( {x}^{*} [ - n ] * x [ n ] ) w [ n ] ) \vert \vert FFT ( ( y [ - n ] * y [ n ] ) w [ n ] ) \vert}$$

In particular, the modulus was used due to the linear phase introduced by the fast Fourier transformation employed in order to compute the DFT (which assumes causal sequences).

Coherence is a random process, and the coherence C_{xy} is related to a correlation coefficient and therefore follows the same distribution. As a consequence applying a Fisher z-transformation will normalize the process:

zᵢⱼ=tan h⁻¹(|γᵢⱼ(ω)|)

Based on this transformation, it is possible to compute confidence limits for Cᵢⱼ:

tan h(zᵢⱼ−b−σ_{z}Z_{0.5α})≦γ≦tan h(zᵢⱼ−b+σ_{z}Z_{0.5α})

where Z_{α} is the 100α percentage point of the normal distribution and

$$b = \frac{p}{n - 2 p}$$

p is the number of input processes that are linearly combined to obtain a process y. Here, with one input and one output, p=1 and b=(n−2)⁻¹ (where n is the number of degrees of freedom). In this example the size of the sample was approximately 1000 for 8.3 h of sleep.

Due to the fact that d.f.>>2, b=n⁻¹, For α=0.05 one gets with Z_{0.025}=−1.9599 and

$${σ}_{z} = \sqrt{\frac{1}{n}} ( 1 - {0.004}^{1.6 {γ}_{ij}^{2} + 0.22} )$$

$$tanh ( {z}_{ij} - \frac{1}{N} - 1.96 {σ}_{z} ) \leq γ \leq tanh ( {z}_{ij} - \frac{1}{N} + 1.96 {σ}_{z} )$$

As an example having Cᵢⱼ=0.8, one gets the 95% confidence interval:

$$tanh ( {tanh}^{- 1} ( \sqrt{0.08} ) - \frac{1}{1000} - 1.96 \sqrt{\frac{1}{1000}} ( 1 - {0.004}^{1.6 * 0.8 + 0.22} ) ) \leq γ \leq tanh ( {tanh}^{- 1} ( \sqrt{0.08} ) - \frac{1}{1000} + 1.96 \sqrt{\frac{1}{1000}} ( 1 - {0.004}^{1.6 * 0.8 + 0.22} ) )$$

### REM Density

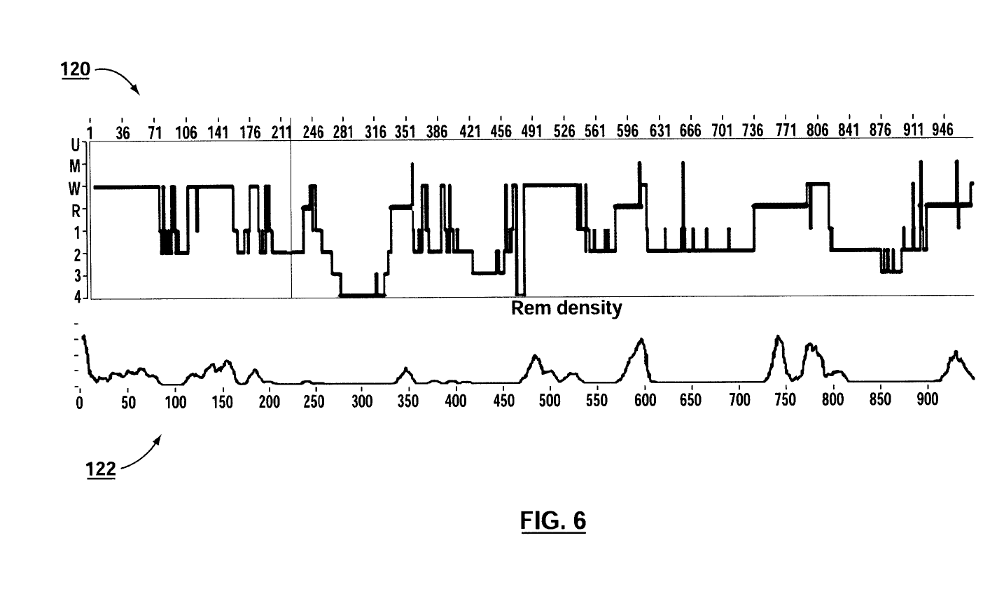

Turning now to FIG. 6, illustrated therein is an exemplary diagram of an estimate of REM density according to one embodiment.

In general, a REM density estimator may work in conjunction with a sleep analyzer module. In particular, the REM density estimator can detect the rapid eye movement (REM) of a patient during sleep. This result can be refined later on using sleep staging information.

In some cases, all of the REMs detected during stages other than stage 5 (REM sleep) will be discarded (i.e., any detected rapid eye movements associated with sleep in stages 1-4 will be ignored), which should help provide for a more accurate determination of REM density.

In some cases, the data is then filtered with a band-pass filter with pass band boundaries (0.5, 10 Hz) and a notch filter, so as to create a zero-mean time-series.

FIG. 7 shows a schematic diagram of some functional components of a REM density estimator 130 according to one embodiment. In particular, this embodiment includes a first digital filter 132 that is coupled to a segmentation module 134. The REM density estimator 130 also includes a synchronization analyzer 136, and is coupled to a second digital filter 138.

In some cases, the input channels for the REM density estimator 130 are either Electro-oculogram channels (EOG) or Fronto-Parietal (FP) EEG channels. Eye movements will normally produce opposite polarity signals in the two EOG channels. Confounding frontal slow activity will either have same polarity or misaligned waves in the two EOG channels.

The segmentation module 134 is adapted to identify candidate wavelets. The synchronization analyzer 136 then retains those candidates that are aligned in opposition on the two EOG channels.

The segmentation module produces two series of vectors of the form:

REMvUDᵢ[k]=[A1 d11 d12 t]^{T}

SYNCvᵢ[k]=[v1 v2 v3]^{T}

REMvUD contains important morphological characteristics of wavelets: amplitude, duration of first half (d11), second half (d12) and time of occurrence (t). The input time series for segmentation are all zero-mean.

For this particular example, the noise level in the study was first estimated, and then the index set was built. Then an operator was defined that finds the zero crossings of a time series x[n]:

zx=Zero(x)={n|x[n−1]*x[n]≦0}

Defining the derivative operator D as:

Dx=x[n]−x[n−1]

and using the operators D and Z, the following random processes can be built:

$${n}_{δ} = {\sum }_{i} ( {z}_{x} [ i ] - {z}_{x} [ i - 1 ] \geq \frac{{f}_{s}}{4} ) ( {z}_{x} [ i ] - {z}_{x} [ i - 1 ] \leq {f}_{s} ) \frac{{z}_{x} [ i ] - {z}_{x} [ i - 1 ]}{{f}_{s}}$$

which is actually counting the waves that have a frequency in the delta range (i.e., 1-4 Hz). The set was then built:

zdₓ=Zero(Dx),

along with the set:

A={x[zdₓ[n]]−x[zdₓ[n−1]]|zdₓ[n]]−zdₓ[n−1]]<=0.2fₛ}

Let: N=card(A). The rank operator is then defined:

A□ₚW[n]=p th rank of {A[0] . . . A[N]}

where W is a window W=(0 1 . . . card(A)). Let p=0.9*N, then define the noise:

noiseA=A□ₚW[n]

Setting the amplitude threshold:

$$thr = \{ \begin{matrix}2 * noiseA ; & 2 * noiseA > 20 \\ 20 & otherwise\end{matrix} \}$$

allows the following set to be built:

zₓ=Zero(x),

M=max(x); xε[zₓ[n−1],zₓ[n]],nε[1,card(zₓ)]

m=min(x); xε[zₓ[n−1],zₓ[n]]

A vertex direction can then be defined:

Vup=M>|m|?true:false;

In general, a wavelet is pointing up if between two consecutive crossings of the baseline, a maximum point is larger than the absolute value of a minimum point. This property is true due to the zero-mean property of the time-series. Usually the most accurately identifiable point of the triple (Vᵢ Vᵢ₋₁ Vᵢ₊₂) is the vertex (Vᵢ₊₁).

A wavelet can be modelled by a triangle (Vᵢ Vᵢ₊₁ Vᵢ₊₂), and the wavelet parameters are the signed amplitude and the durations of the half-wavelets:

A1=x[zₓ[i+1]]−x[zₓ[i]]

d11=10̂3*(zₓ[i+1]−zₓ[i])/fₛ

d12=10̂3*(zₓ[i+2]−zₓ[i+1])/fₛ

t=zₓ[i];

A candidate wavelet is detected when the characteristics meet certain criteria:

REMvUDₖⱼ={[A d11 d12t]ₖⱼᵢ^{T}|d11<d12;d11+d12>200;A>thr}

REMvUDₖᵢⱼ represents the characteristic vector for REM “l” in epoch “j” on channel “k”. A second set can then be built:

SYNCv_{kJ}={[zₓ[i]zₓ[i+1]zₓ[i+2]]ₖⱼᵢ^{T}|d11<d12;d11+d12>200;A>thr}

where SYNCvₖⱼᵢ represents the synchronization vector for REM “l” in epoch “j” on channel “k”.

FIG. 7a shows an example of REM activity on EOG channels. For instance, the synchronization analyzer takes the sets SYNCvₖ where k={1,2} on the two EOG channels and correlates their position as follows:

$${REM}_{j} = \{ t \vert StageREM [ j ] * {SYNCv}_{1 ji} [ 2 ] * ( \vert \vert {SYNCv}_{1 ji} - {SYNCv}_{2 jm} \vert < 100 ) * ( {REMvUD}_{1 ji} [ 0 ] {REMvUD}_{2 jm} [ 0 ] < 0 ) * ( \frac{{REMvUD}_{1 ji} [ 0 ]}{{REMvUD}_{2 jm} [ 0 ]} < 4 ) * ( \frac{{REMvUD}_{2 jm} [ 0 ]}{{REMvUD}_{1 ji} [ 0 ]} < 4 ) \}$$

The indices are as follows: j (epoch) l, m (index within epoch for channels 1 and 2 respectively)

StageSREM is a boolean function that is true if the epoch is part of a REM stage. The stage may be provided by a stager module (not shown).

Each epoch has a set {REMⱼ} of times where a REM occurred. In this case, the whole study has a set of sets of REMS; one REM set for each epoch “j” {REMⱼ}, REMⱼ is a set of REMs in epoch “j”.

One can estimate the REM density in multiple ways depending on the desired purpose. For instance, a rolling window of variable duration may be used, depending on the length of the REM episode.

$$RD [ k ] = \frac{{\sum }_{i = - \frac{M}{2}}^{\frac{M}{2}} StageREM ( k - i ) * Card ( {REM}_{k} )}{{\sum }_{i = - \frac{M}{2}}^{\frac{M}{2}} StageREM ( k - i )}$$

Setting M=1, one gets the REM count per epoch. Setting M to sup(Card(REMᵢ)), where sup stands for supremum, one gets the average REM count per REM episode, where the duration of the REM episode can be anything between 1 and 200 epochs.

### Transformer

Various factors that can influence the architecture of sleep include the gender and age of a patient. For example, information about the evolution of normal sleep with age and gender can be obtained from various sleep clinics, such as the Sleep and Alertness Clinic (Toronto), and is generally discussed as the ontogeny of sleep stage percentage.

Before classification by a diagnosis system, it may be beneficial to try to compensate for this variable bias (for example using the transformer 92 shown in FIG. 4) to at least partially mitigate the effects of gender, age, and so on. In order to correct for some such variability and distinguish pathognomonic signs, the following transformation of the sleep markers was adopted SM={TS1, TS2, TSD, TREM}. The initial T or TS reads total and total stage respectively.

$$SM = F * \frac{( SM - \overline{{SM}_{F} )}}{{SM}_{F}} + ( 1 - F ) * \frac{( SM - {SM}_{M} )}{\overline{{SM}_{M}} )}$$

Where SM_{F} bar represents average sleep marker for females of the age group that bracket test cases. For example, for a female patient, age 45, with 30% S2 we would obtain for SM=TS2:

$$TS 2 = 1 * \frac{( 30 - 54 )}{54} + ( 1 - 1 ) * \frac{( 30 - 54.75}{54.75 )} = - 0.44$$

The units after normalization are in the range [−1, 1], where negative values are for cases with less than normal average sleep markers, and positive values represent values that are above normal. The absolute values of SM variables are generally in the range [0, 1].

Some classification methods include parameters that have close ranges and similar variance. This is the case for multivariate distance calculations.

Other parameters were normalized due to largely different ranges as follows: sleep efficiency (SEF), arousal index (ARI), sleep onset (SO), REM latency (REM_LAT), apnea-hypopnea index (AHI), periodic leg movements (PLMS), age (AGE), number awakenings (NUM_AWA), lights out to sleep onset (LOSO), total sleep time (TST), wake after sleep (WAS), sleep period time (SPT) as follows:

SEF=SEF/100;

ARI=ARI/100.0;

SO=SO/100.0;

REM_LAT=REM_LAT/120.0;

AHI=AHI/100.0;

PLMS=PLMS/100.0;

AGE=AGE/100;

NUM_AWA=NUM_AWA/100;

LOSO=LOSO/100;

TST=TST/1000;

WAS=WAS/1000;

SPT=SPT/1000;

At this point all parameters have been calculated and normalized and one can proceed to classification methods.

### Classification

Before discussing the classification step in greater detail, it may be helpful to review some of the above described teachings.

In particular, a set of microarchitectural parameters may be calculated that result from ultradian rhythm relationships. These parameters can then be adjusted for bias and variance.

Furthermore, a set of biological markers can be extracted based on sleep architecture and a set of sleep continuity indicators (which may be normalized). All absolute values can be normalized within the range [0, 1], thus setting the stage for multivariate classification in a [−1, 1] hypercube.

In general, there are numerous ways of classifying multivariate data. The common denominator is that they are all statistical in nature. The next task is thus a binary classification problem, to answer the question: is the multivariate test vector in class A (normal) or B (depressed)?

One of the ways to solve the classification task is by using an artificial neural network. A brief discussion of neural networks is provided herein, although it will be appreciated that neural networks are incredibly complex and powerful and a detailed discussion is beyond the scope of this document.

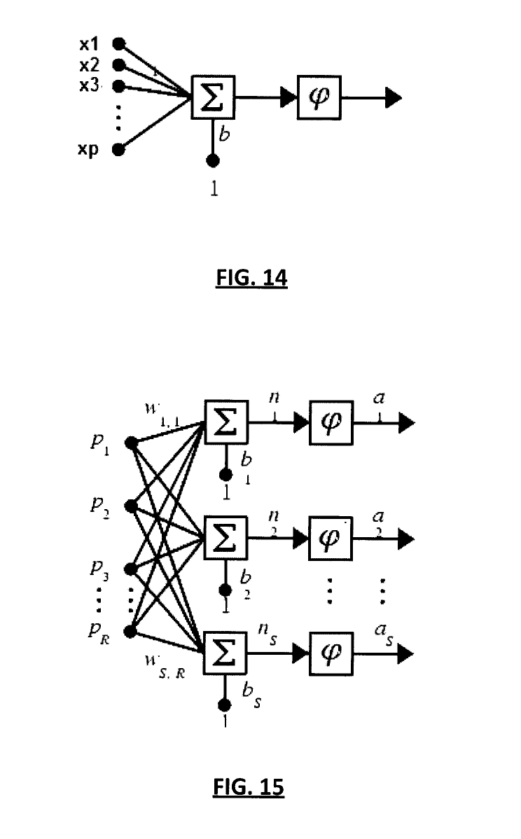

In general, an artificial neural network is a machine that is designed to model the way the brain performs a particular task. A neural network is formed by using artificial neurons connected by synapses in ways mimicking the biological neuronal network model. Examples of a model artificial neuron and artificial neural network are shown in FIGS. 14 and 15, respectively.

In general, artificial neurons are computational units that have a variable number of input synapses that permit them to connect to other neurons in a network. The set of synapses of a neuron forms the receptive field of the neuron. A synapse is characterized by its strength and is modified by exposing the network to training patterns. Synapses can be inhibitory or excitatory. Artificial neural networks are therefore considered to be knowledge encoders. Knowledge is information used by the network to respond to exterior stimuli applied to its receptive field.

The synaptic inputs may be summed in an accumulator which is the mathematical equivalent of the soma, or cell body of biological neurons. Thus, the artificial neuron acts as a linear combiner:

$${v}_{k} = {\sum }_{i = 1}^{p} {w}_{ki} {x}_{i}$$

The output of the linear combiner is called induced local field or activation potential.

The other ingredient of a neuronal model is the activation function, which limits the output of the neuron to a finite value, thus making the neuron a nonlinear computational element. For example, the function implemented by a single neuron may be modeled as:

$${y}_{k} = ϕ ( {\sum }_{i = 1}^{p} {w}_{ki} {x}_{i} + {b}_{k} )$$

where bₖ is a bias, and if present can shift the input of the neuron up or down depending on its value.

Various kinds of activation functions may be used as are generally known, such as sigmoid, hyperbolic tangent, and a Heaviside function

$$ϕ ( v ( n ) ) = atanh ( bv ( n ) )$$

$$ϕ ( v ) = \frac{1}{1 + exp ( - av )}$$

In general, the hyperbolic tangent and the sigmoid functions are continuous and therefore differentiable whereas the Heaviside function is not.

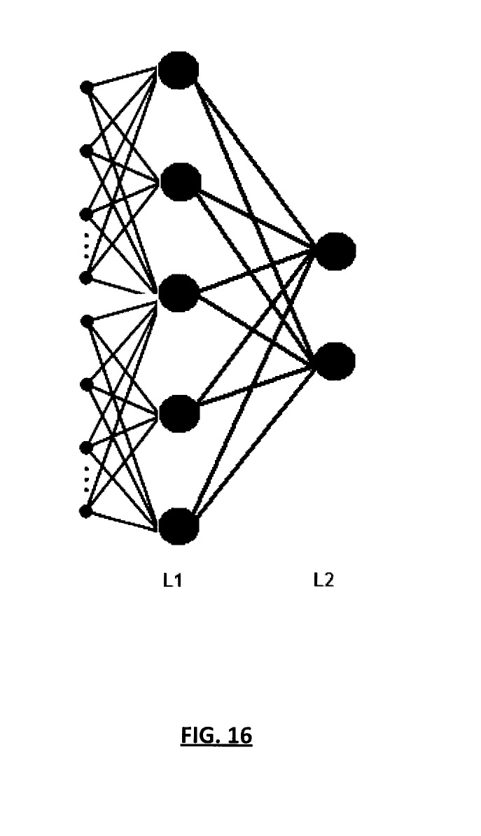

One specific example of a fuzzy logic method that may be implemented will now be described. In this embodiment, a multilayer feedforward artificial neural network was created with one hidden layer and one output layer, also commonly called a multilayer perceptron and as generally shown in FIG. 16.

This type of neural network is called a perceptron due to the presence of the nonlinear activation function, and this type of network learns with a teacher. In particular, the repeated presentation of training examples produces an error signal at each neuronal output from the output layer.

eⱼ(n)=dⱼ(n)−yⱼ(n)

The error signal is the difference between the desired output (d) and the actual output (y) at each time step (n).

Assuming a batch mode of training the average error energy may be computed as:

$$\overline{ɛ} = \frac{1}{2 N} {\sum }_{n = 1}^{N} {\sum }_{W}^{ } {( {e}_{j} ( n ) )}^{2}$$

The double summation is over all the synaptic weights (W) and all presentations of training patterns (N). The adjustment of weights may be done in a direction opposite to the gradient of the error energy. This adjustment has the effect of decreasing the error energy and therefore bringing the output closer to the desired response:

$$Δ {w}_{ij} = - η \frac{\partial \overline{ɛ}}{\partial {w}_{ij}}$$

The weight adjustment is generally done only after the network has been presented the whole set of training patterns. This equation can thus be expanded using the chain rule of differentiation and specifying the form for the activation function. In particular, the learning rate η can be adjusted as the number of iterations increases.

The algorithm for training this network is general as follows

1. Initialize Network

Set the weights to values picked from uniform distribution with zero mean and variance, in order to set the standard deviation of induced fields of neurons to be above the linear part and below the saturation part of the activation function. A simple and popular choice is initialization of weights from a uniform distribution is between −1 and 1.

Wᵢⱼ=rand(−1,1)

2. Train the Network: Forward Pass

Compute starting at the input layer, for each neuron the output using linear combiner equation above. When all outputs of first layer are available, compute the output of the second layer using as input the output from the previous layer.

$${v}_{j}^{l} ( n ) = {\sum }_{i = 0}^{m} {w}_{ji}^{l} {y}_{i}^{l - 1}$$

where L is the layer number, j neuron from layer l, yᵢ input on synapse l of neuron j. The error between desired output and actual output on neuron j is then:

e(n)=dⱼ(n)−φ(vⱼ(n))

3. Train the Network: Error Back-Propagation

Take the error from the output layer of neurons and propagate toward the input in order to redistribute the blame for error among the neurons of the network. To do this, the gradients or the error energy should be computed:

$$∇ \overline{ɛ} = \frac{\partial ɛ ( n )}{\partial {w}_{ji} ( n )}$$

Then the synapses can be updated:

$$Δ {w}_{ji} = - η \frac{\partial ɛ ( n )}{\partial {w}_{ji} ( n )}$$

and the local gradients computed for neuron j:

$${δ}_{j} ( n ) = \frac{\partial ɛ ( n )}{\partial {v}_{j} ( n )}$$

There are distinct cases for neuron j being an output neuron (L2) or a hidden neuron (L1):

$${δ}_{j}^{l} ( n ) = \{ \begin{matrix}{e}_{j}^{l} ( n ) \frac{\partial {ϕ}_{j}}{\partial {v}_{j}^{l} ( n )} ; & j ∈ {L}_{2} \\ \frac{\partial {ϕ}_{j}}{\partial {v}_{j}^{l} ( n )} {\sum }_{k ∈ L 2} {δ}_{k}^{l + 1} {w}_{kj}^{l + 1} ( n ) ; & j ∈ {L}_{1}\end{matrix}$$

For the activation potential of neuron j in layer l, one then arrives at:

$$\frac{\partial {ϕ}_{j}}{\partial {v}_{j}^{l} ( n )} = \frac{b}{a} ( a - {y}_{j}^{l} ( n ) ) ( a + {y}_{j}^{l} ( n ) )$$

Combining these equations allows the local gradient of neuron j in layer l to be determined:

$${δ}_{j}^{l} ( n ) = \{ \begin{matrix}\frac{b}{a} ( {d}_{j} ( n ) - {y}_{j} ( n ) ) ( a - {y}_{j} ( n ) ) ( a + {y}_{j} ( n ) ) ; & j ∈ {L}_{2} \\ \frac{b}{a} ( a - {y}_{j} ( n ) ) ( a + {y}_{j} ( n ) ) {\sum }_{k ∈ L 2} {δ}_{k}^{l + 1} {w}_{kj}^{l + 1} ( n ) ; & j ∈ {L}_{1}\end{matrix}$$

4. After all Test Examples have been Exhausted Update all the Weights for all the Neurons Using the Stored History of Partial Derivatives from all Training Examples:

$$Δ {w}_{ji}^{l} = - \frac{η}{N} {\sum }_{n = 1}^{N} {y}_{i} ( n ) {δ}_{j}^{l} ( n )$$

In this equation yᵢ is the input signal to neuron j on synapse l at time n.

Using this approach, there are generally two passes of the computation for each training example: the forward pass, where the information is propagated through the network and no modification is made to the synaptic weights, and the backward pass, where the error signal between the desired response and the actual response is redistributed in the network and corrections are made to the synapses based on the blame assigned to each neuron.

Various optimizations and training algorithms are generally possible.

For example, gradient descent with momentum sues a modification of the update rule for synaptic weights based on previous updates:

Δwⱼᵢ(n)=αΔwⱼᵢ(n−1)+ηδⱼ(n)yᵢ(n)

The momentum constant α has the role to avoid network instability and has an absolute value between 0 and 1. It can be proven, by solving the difference equation, that for consecutive, same direction variation of the weight vector accelerates the descent while for alternating sign changes it decelerates the descent on the error surface, thus stabilizing the learning. Practically this is not necessarily so. The momentum constant is a new problem dependent parameter that doesn't seem to solve anything.

A Riedmiller algorithm has the advantage that besides adjusting the learning rate it eliminates the dependence on the partial derivative of the error energy which can be unexpected and therefore the whole adaptation of the learning rate is vacuous.

In particular, the following values may be computed:

$${Δ}_{ij} = \{ \begin{matrix}{η}^{-} {Δ}_{ij} ( n - 1 ) & if \frac{\partial ɛ}{\partial {w}_{ij}} ( n ) \frac{\partial ɛ}{\partial {w}_{ij}} ( n - 1 ) < 0 \\ {η}^{+} {Δ}_{ij} ( n - 1 ) & if \frac{\partial ɛ}{\partial {w}_{ij}} ( n ) \frac{\partial ɛ}{\partial {w}_{ij}} ( n - 1 ) > 0 \\ & {Δ}_{ij} ( n - 1 )\end{matrix}$$

This equation may then be used to update the synaptic weights:

$$Δ {w}_{ij} = \{ \begin{matrix}- {Δ}_{ij} & if \frac{\partial ɛ}{\partial {w}_{ij}} ( n ) > 0 \\ {Δ}_{ij} & if \frac{\partial ɛ}{\partial {w}_{ij}} ( n ) < 0 \\ & 0\end{matrix}$$

In this equation, weights are decreased if the error is growing (partial derivative positive) and increased if the partial derivatives are negative.

In this approach, these equations are computed at the end of each epoch, when all training patterns have been presented to the network. The next epoch then uses the adapted values. Then another adaptation takes place and so on and so on.

For each epoch, the data can be transformed to zero mean and standard deviation 1:

$$y = \frac{x - \overline{x}}{\sqrt{\frac{1}{N} {\sum }_{1}^{n} {( {x}_{i} - \overline{x} )}^{2}}}$$

Next, one can de-correlate the inputs because correlations will induce preferential learning directions. In order to achieve this goal, one can use the Karhunen-Loeve transform (KL). The KL transform finds linear combinations of input variables that have maximal variance and zero covariance. This step will both reduce the redundancy of the variables by eliminating low variance components and eliminate preferential learning directions. The KL transform is obtained by projecting input vectors on the eigenvectors or the covariance matrix.

In some cases, the low variance directions should be removed at the 0.01 level.

The classification of a test vector is accomplished after applying the same transformation to the test vector that was applied during training, namely the test vector may be projected on the principal directions of the training covariance matrix.

Generally, performance is influenced by network configuration, complexity of the problem and adequacy of the training set. In some cases, it may be beneficial that the network configuration should be the simplest that is capable of solving the problem.

One practical rule for selecting the number of training patterns to achieve a good generalization performance is O (W/ε), where W is the number of synapses in the network and ε is the maximum percent error accepted. (e.g., for 4 input parameters, 7 neurons in the hidden layer and 2 output neurons one gets W=4*7+7*2=42 N=42/0.1=420).

By trial and error a network with 7 hidden neurons and 2 output neurons was identified as being suitable for our application. The receptive field of the sensory neurons in the hidden layer was variable between 2 and 36 inputs, depending on which parameters were discarded in our trials. The results are presented in the discussion section below.

It will be appreciated that in general, various other classification techniques may be used accordingly to the teachings herein, and will not be discussed in detail. For example, it may be possible to use a two layer neural network which has Radial Basis Function (RBFNN) neural network as a first layer. A RBFNN is a three layer neural network that has a layer of sensory neurons, a hidden layer and a set of output neurons. This type of network solves the classification problem by treating the problem as a function fitting problem in high dimensional space.

Other types of neural networks that might be suitable for classification include Probabilistic Neural Networks (PNNs), and Support Vector Machines (SVMs).

In some embodiments, it may be possible to use combinations of weak models to obtain performance comparable to strong learning models using committee machines. For example, one approach called bagging uses model averaging, where a number of learning machines (experts) would be trained to solve the classification problem. Other techniques include boosting by filtering, the AdaBoost algorithm, CART (classification and regression trees), using a committee of logistic experts, using mixtures of experts (ME), and using a hierarchical mixture of experts (HME).

In the particular classification problem being faced here, the classifier must decide whether a vector x is from class C₁ or C₂. The uncertainty that characterizes the problem is summarized by the joint probability density p(Cᵢ, x), which is commonly known as inference. Once the inference step is complete, decision theory can be applied to solve the classification problem.

Given a vector x, one would like to determine if a particular patient is depressed or not based on an available a training sample. Using Bayes' theorem, the posterior probability can be determined as:

$$p ( {C}_{i} \vert x ) = \frac{p ( x \vert {C}_{i} ) p ( {C}_{i} )}{p ( x )}$$

In the particular case we are interested in, namely a binary classification problem, p(Cᵢ) represents the prior probability for class Cᵢ with the probability to observe x:

p(x)=p(x|C₁)+p(x|C₂)

At the same time we have the joint probability:

p(x,Cᵢ)=p(x|Cᵢ)p(Cᵢ)

If a prior p(Cᵢ) is available, then one can get a revised posterior probability due to the addition of the new information due to the latest test.

In general, the determination as to whether a patient is depressed or not may be based in the maximum posterior probability.

Another aspect in decision theory is the minimization of the cost due to error. This theory provides techniques for considering the risk associated with misclassification. In particular, the prevalence of disease in the population and asymmetrical risk associated with false positives and false negatives must be considered.

More specifically, if population sample where the normal state is prevalent is used to train a diagnostic system, then this data will potentially undersample the population of diseased cases and therefore provide incomplete learning. Balancing the training populations, on the other hand, may create false priors due to the exaggerated presence of disease states within the sample set. Corrections should be made to adjust these priors to provide a training sample that generally accurately reflects the distribution of depression within a population.

For example, one can introduce a loss function L which is the overall cost due to the incurred decisions. In some cases the goal is to reduce and even minimize E(L) by finding the regions Rᵢ that best accomplish that aim:

$$E [ L ] = arg {max}_{{R}_{i}} {\sum }_{i} {\sum }_{j} {\int }_{{R}_{i}} d {xL}_{ji} p ( x , {C}_{j} ) = {\sum }_{j} {L}_{ji} p ( {C}_{j} \vert x )$$

In this equation, Rᵢ is decision region for class Cᵢ given the example is from class Cⱼ. The second equality in this equation results from Bayes' theorem and observing that p(x) doesn't participate in the maximization.

This can be done by knowing the posterior probabilities. In particular, priors p(Cᵢ) can be computed from the training set as well as the class conditional densities. Decisions can then be made by using the maximum posterior probability criterion.

In some cases, the cost function can be changed on the fly, for instance based on application once the posterior probabilities were determined. In a clinical situation it may be more important to increase the sensitivity of the test for screening purposes, knowing that if a false positive is returned, more tests may be done to increase the specificity of the overall diagnostic (and which can correct for a false positive).

In another setting, if the clinician has already some evidence of the existing disease and wants confirmation from complementary tests, then the clinician may choose to balance the cost in favor of specificity.

In addition, in some cases decision zones with lower than desired posterior probability can be excluded. For instance, in cases where posterior probability is lower than a threshold can be considered as undecided.

In some cases, mixed information from different sources can be divided and treated separately. The results can then be combined using probability theory. For example the parameters stemming from the microarchitecture of sleep could be used independently (more or less artificially) from the more conventional sleep markers. In this case, the results may be combined, during training, in class conditional joint probabilities:

p(xₘ,x_{c}|C₁)=p(xₘ|C₁)p(x_{c}|C₁)

In general, the posterior probability can be used to reach a decision:

$$P ( {C}_{i} \vert {x}_{m} , {x}_{c} ) = \frac{P ( {C}_{i} \vert {x}_{m} ) P ( {C}_{i} \vert {x}_{c} )}{p ( {C}_{i} )}$$

The priors P(Cᵢ) can be estimated from the proportion of the data pertaining to each class in the training sample (assuming random sampling).

To reach a final determination on classification, various techniques may be employed. For example, one form of classification doesn't require estimation of posterior probabilities and estimates the input-output relationship directly. A popular method minimizes the least square error between the model and the desired output. The simplest discriminant (linear discriminant) builds a D−1 dimensional hypersurface in a D dimensional decision space.

In other cases, probabilistic models based on maximum posterior probability may be used. Furthermore, it may also be possible to use k Nearest Neighbour (kNN) approach. The kNN approach has some very nice features that make it desirable in some applications. Among the advantages are the independence on distribution of the data in the decision volume, furthermore, this method is not disturbed by the uneven density of training data in high dimensional spaces, a problem known as curse of dimensionality. Another advantage is that the error of the method is never worse than twice the minimum achievable error rate.

Various other techniques for reaching a final determination on classification will be appreciated based on the teachings herein.

### Various Alternative Embodiments

In general, the teachings herein may be used in various different embodiments that may be useful for diagnosing depression.

For example, in one embodiment, the teachings herein may be implemented in stand-alone software. In particular, diagnostic software application may be provided that can complement existing polysomnographic equipment, for example as used in sleep laboratories and other medical facilities. In some such cases, the diagnostic software may be implemented using existing hardware, such as a processing device already present in a sleep laboratory.

In general, assuming that sleep laboratories have equipment that is adequate for recording EEG, the teachings herein may be useful to provide enhanced diagnostic methodology for sleep that can help diagnose depression.

In some embodiments, the teachings herein may be used to provide an extra analysis of the sleep record and functions as a screening tool for depressed people in a sleep laboratory. This is in agreement with the relatively well known fact that about 20-30% of the patients seen for sleep disorders in the sleep laboratory are depressed and should be diagnosed and treated accordingly.

In another embodiment, the teachings herein could be used to provide a software application for use in a patient's home. This may include using a headbox that can be sent to a patient's home, and can be used in combination with an EEG review station existing remotely at the point of care.

More particularly, currently the clinician in a sleep laboratory will analyze sleep stages using a well-established method that includes relatively precise positioning of the electrodes on the scalp of a patient.

In a patient's home, there are several barriers to this approach. First, it is generally not possible (or at least may be difficult) for a patient to apply the electrodes to his or her own scalp. Furthermore, most patients will likely lack anatomical knowledge that is necessary to achieve standard electrode placements on a scalp.

Moreover, an additional barrier is one of interpretation. In particular, namely replacing the standard electrode arrangement that a clinician would use means that one can no longer reliably use the textbook approach for interpretation and thus would normally be unable to produce a reliable diagnostic based on the standard set of rules. More specifically, these rules tend to be become highly unusable as electrode placement varies, since these are tightly bound to the recording technique.

Some of the teachings herein are directed to new methods that may overcome at least some of these difficulties, particularly for the home setting, and yet still and provide results that are at least comparable to results obtained with established methodologies. In particular, these new techniques may be much more robust to electrode placement error, more consistent across a population of subjects, and more amenable to application by the patient himself or herself (i.e., using a net).

Furthermore, the teachings herein may provide diagnostic systems that capable to make use of existing EEG equipment in combination with modified methodology and analysis tools.

These implementations may result in reduced costs, and in some cases allow for the elimination of redundant equipment in a sleep laboratory.

The teachings herein may have other applications, for example for performing home diagnostics for sleep issues more generally in addition to depression. This may potentially extend the boundaries of sleep laboratories and permits screening of depression in wide geographic areas, including in remote areas.

In some cases, the teachings herein may empower a psychiatrist, general practitioner, or sleep specialist to do screening tests for depression, thus improving quality of life for patients, potentially reduce cost to society and health care systems alike. In particular, a psychiatrist may be provided with quantitative tools that can provide for more ample openings into metal health fields based on scientific methods that describe generally repeatable methodologies that are amenable to standardization.

In some other embodiments, the teachings herein may be directed to a hardware solution which may be particularly suitable for general practitioners, psychiatrists and the like who may not currently have EEG equipment. Purchasing high-end EEG equipment may be too expensive for many of these practices, particularly due to complexity of operating the equipment, long learning curves and volume in the laboratory.

For these practices a lightweight solution, with minimal footprint and requiring minimal learning required may be provided using at least some of the teachings herein. In one embodiment, the system may include a review station, that a tablet, laptop, personal computer or other computing device. The computing device can be coupled to one more recorder units (headboxes) that can be sent to the patient's home.

The recording device may include a battery powered EEG recorder with minimum number of channels, and which could use a data protocol (i.e., USB, wireless or Internet connectivity) for later retrieval of the data. The recorder may be capable of storing data for a particular minimum number of hours (e.g., 40 hours or more), which may correspond to three or more nights of sleep analysis. Such a home device may be capable of monitoring electrode impedance and recording quality, and may notify the patient to make corrections in order to avoid poor recordings where appropriate.

In some other embodiments, the teachings herein may be directed to OEM module can be provided for manufacturers of EEG and sleep monitoring equipment that would want to extend the capabilities and value of their monitoring solutions. In particular, the teachings herein may be used to develop software application, hardware solutions (or both) that could be integrated with existing EEG and sleep monitoring equipment.

### Discussion of Experimental Results

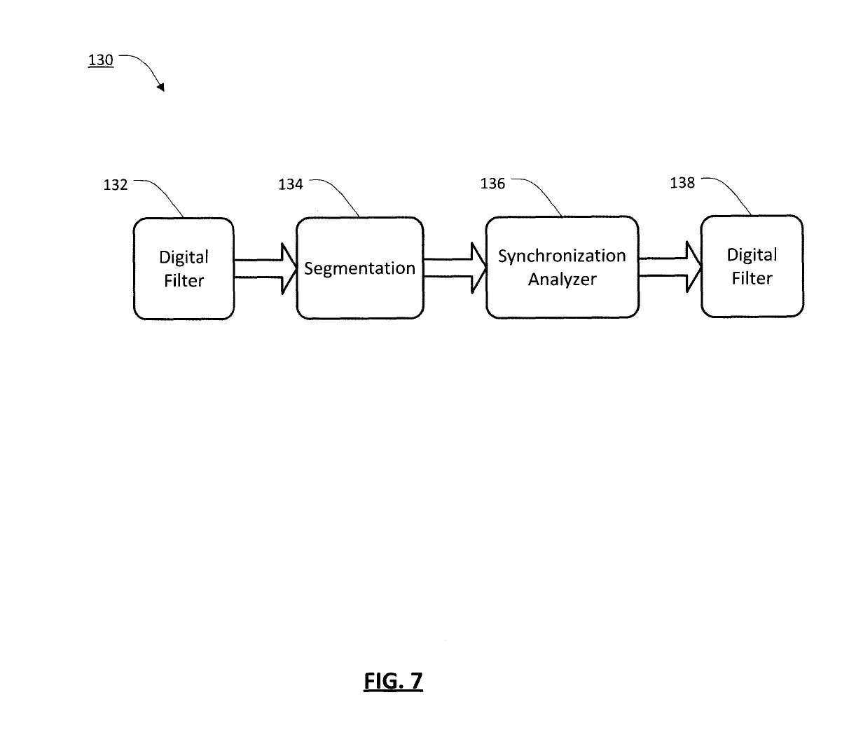

Turning now to FIGS. 8 to 13, various graphs are provided that summarize experimental results using both adult and child patients, and which show that depression leaves a mark on coherence.

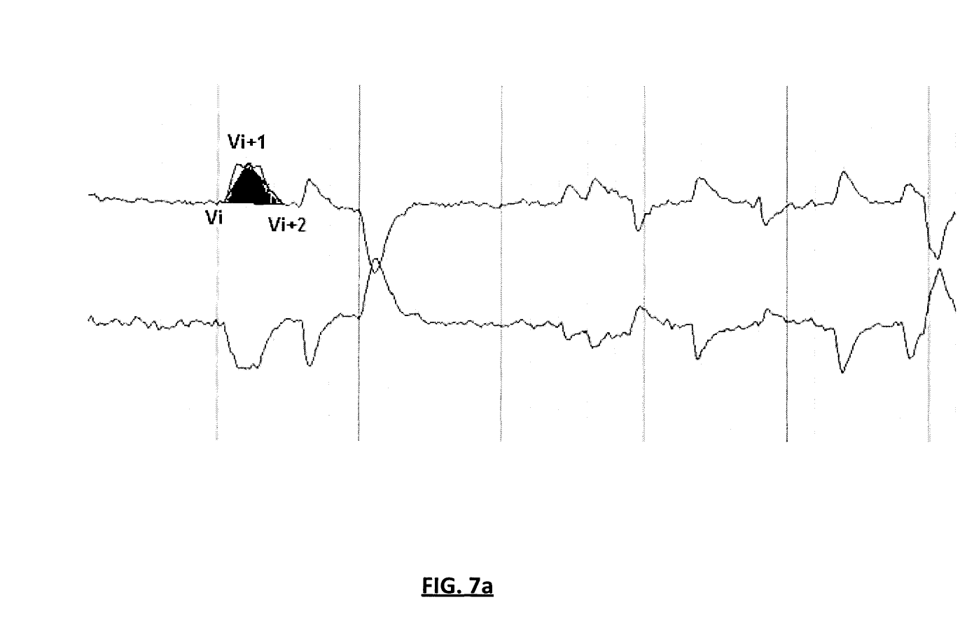

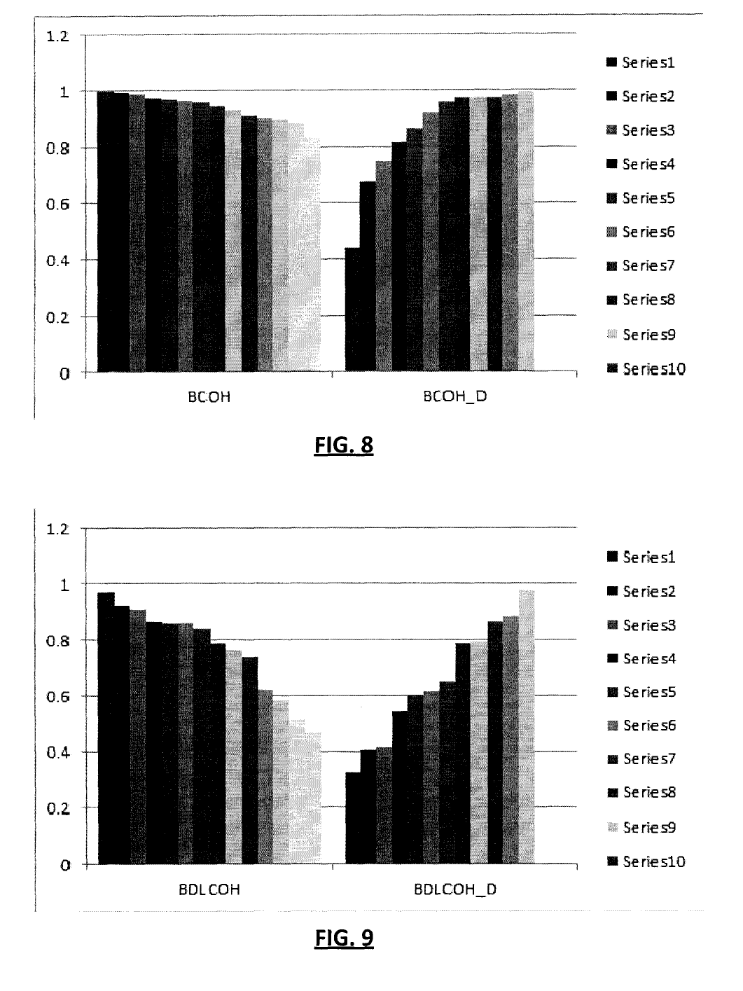

In particular, FIG. 8 is graph comparing beta bilateral coherency for adults between a normal individual (on the left side of the graph) and a depressed individual (on the right side of the graph). FIG. 9 is similar graph comparing beta delta coherency in the left hemisphere, while FIG. 10 is a similar graph comparing beta delta coherency in the right hemisphere.

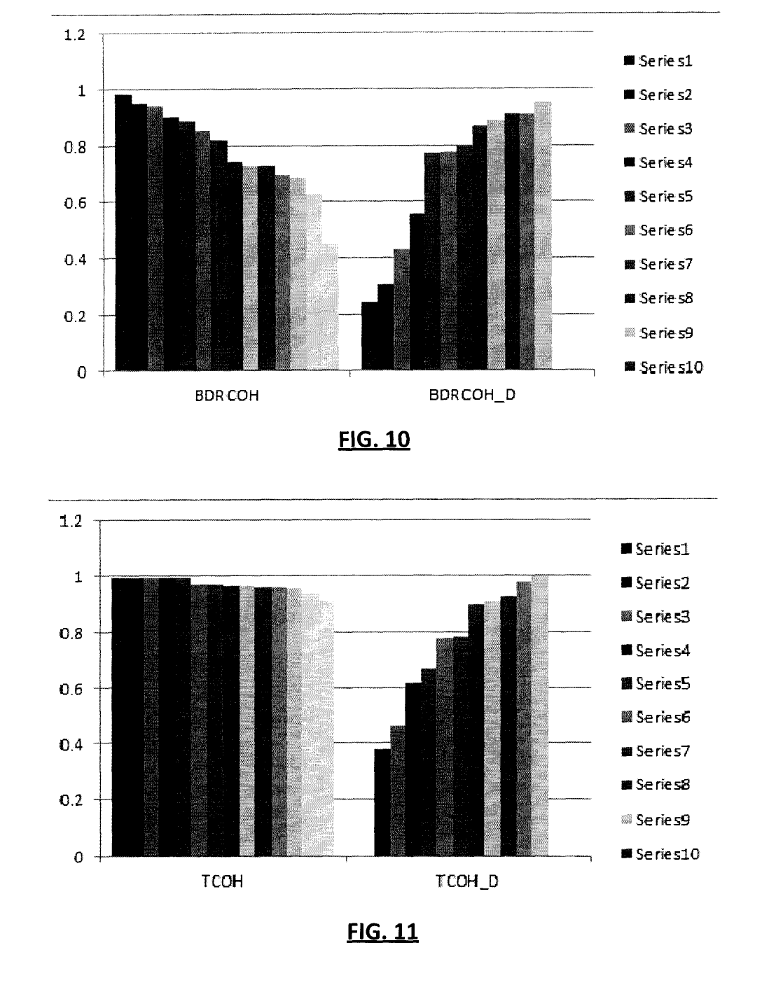

FIG. 11 compares the theta bilateral coherency (TCOH) in adults between a normal individual (again on the left side) with the readings from a depressed individual.

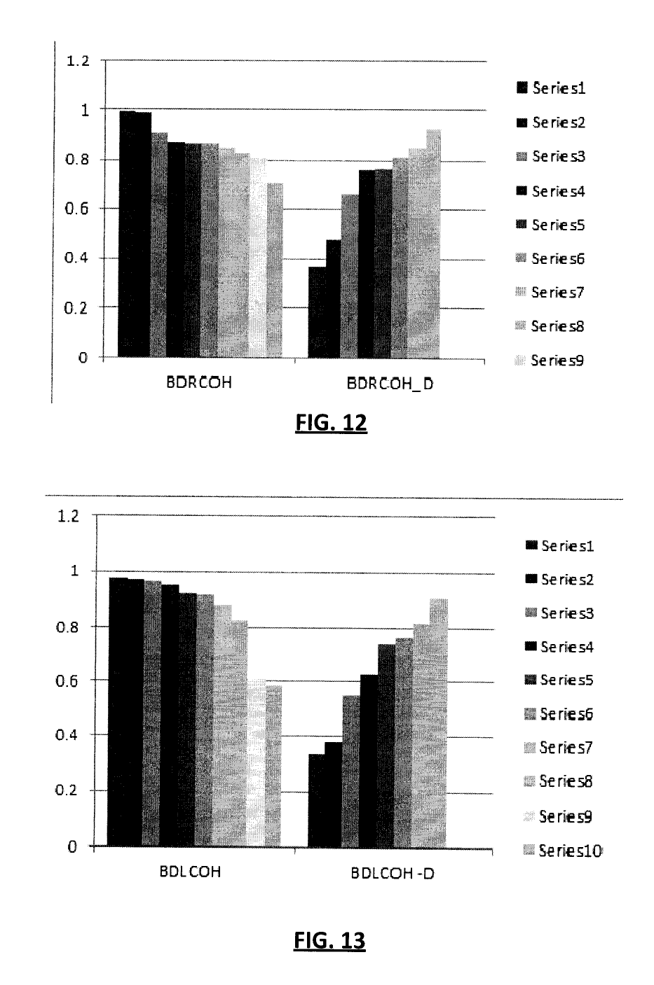

FIGS. 12 and 13 provide graphs of the children studies, comparing beta delta coherency in the right hemisphere (in FIG. 12) and in the left hemisphere (in FIG. 13). Once again, normal individual results are presented on the left, while the results of a depressed individual are shown on the right.

Based on the limited data set for this study, it appears that one particularly suitable parameter for adults is the TCOH with a threshold of 0.95. An effect of age on coherence measures was also observed, since in children the most suitable parameters at present appear to be beta-delta left coherence (BDLCOH) and beta-delta right coherence (BDRCOH).

It appears that that synchronization of the theta component comes later in life at the cost of losing the association of beta and delta rhythms. This can be observed if we compare the normal results between the two groups (children and adults) since the beta and delta rhythms in children are better synchronized in general than in adults. As the children age, this will turn to a stronger theta synchronization.

FIGS. 8 to 13 show that depression has an effect of lowering the coherence in some patients. It remains to be evaluated if different coherence measures are affected in separation or together (e.g. if TCOH is above threshold for a depressed patient, is it possible that BCOH or other coherency measures may be lowered due to an effect due to illness or they always co-vary).

At present, it is believed that estimating the degree of dispersion of the rhythms may be of clinical value. The dispersion in this case is the effect of disease causing the disassociation of rhythms.

In a normal patient it is clear that coherency is very high (above 0.8) at least in some frequency bands, which indicates an almost linear bilateral connection. However, depression breaks this strong linear association (as seen in right hand side of the figures).

It should be mentioned that the rhythms being discussed here are ultradian variations of actual brain rhythms. More specifically, these results are following the component of maximum energy in the variation of some brain rhythm (e.g. theta) energy during the night, and not the brain rhythm itself.

An analogy would be pendulums hanging on a wall. It is generally known that mechanical pendulums hanging on a wall synchronize their rhythms due to vibrations transmitted through the wall. Each clock may indicate a different time but the second is ticking in synchronized manner.

Following this model, it is hypothesized that in the human brain, the brain acts as a synchronizing medium (the wall in the pendulum model) that keeps the clocks (rhythms) aligned or synchronized.

However, disease processes (especially depression) appears to affect the transmission properties of the brain (i.e., changing the rigidity of the wall in our analogy) and hence these “clocks” lose synchronization (i.e., have a lower coherence).

It should also be noted that the loss of linear connection of ultradian rhythms across the brain may be connected to the phase delay observed in REM. The explanation of this can be traced back to the origins of coherency. The coherency is at each frequency a complex number and coherence is the magnitude of coherency. The complex coherency has a spectrum and at a specific frequency it can be interpreted as a correlation coefficient between random processes at that frequency.

In the same manner, interpretation of the complex cross-spectral density h_{xy}(f) at any frequency represents the covariance between component random processes dZₓ(f) and dZ_{y}(f) at that particular frequency.

The spectrum of complex coherency is:

$${C}_{xy} ( f ) = \frac{cov ( {dZ}_{x} ( f ) , {dZ}_{y} ( f ) )}{var ( {dZ}_{x} ( f ) ) var ( {dZ}_{y} ( f ) )} = \frac{{h}_{xy}}{\sqrt{{h}_{xx} {h}_{yy}}}$$

If one expresses the cross-spectral density in polar form we get:

h_{xy}=a_{xy}(ω)e^{iφ(ω)}

Combining these two equations gives a polar representation of complex coherency with phase given by the difference in phase between the two processes, x and y at the given frequency. The denominator phases cancel because the auto-spectral density is real.

If out of all frequencies, one selects the ultradian rhythm, a phase shift (or slipping) can be observed between ultradian rhythms across the brain. In cases of depression, due the modification of the relationship between ultradian rhythms the phase difference is expected to grow.

This slipping can be between same frequencies in the different parts of the brain or same part of the brain at different frequencies. This can be explained by different generators of brain rhythms have different positional relationship relative to the site of electrode recording, and therefore may be affected differently by the interposed brain tissue properties.

At the same time a shift of the REM latency may be observed. REM latency represents the phase of a random process. The random process can be decomposed using the spectral representation in many random processes at different frequencies. Dispersion distorts the form of the sum process and can sometimes manifest as a delay.

In particular, it is helpful to consider how REM latency is observed. A whole night of sleep is represented as a snapshot of the random process that happen life-long. The staging itself is a sort of DPA done manually and relies on the complex interaction of brain rhythms. One can ask the question: is the observed REM shift an effect due to dispersion of ultradian rhythms?

It was noted in the section on microarchitecture that the coherency estimate is positively biased.

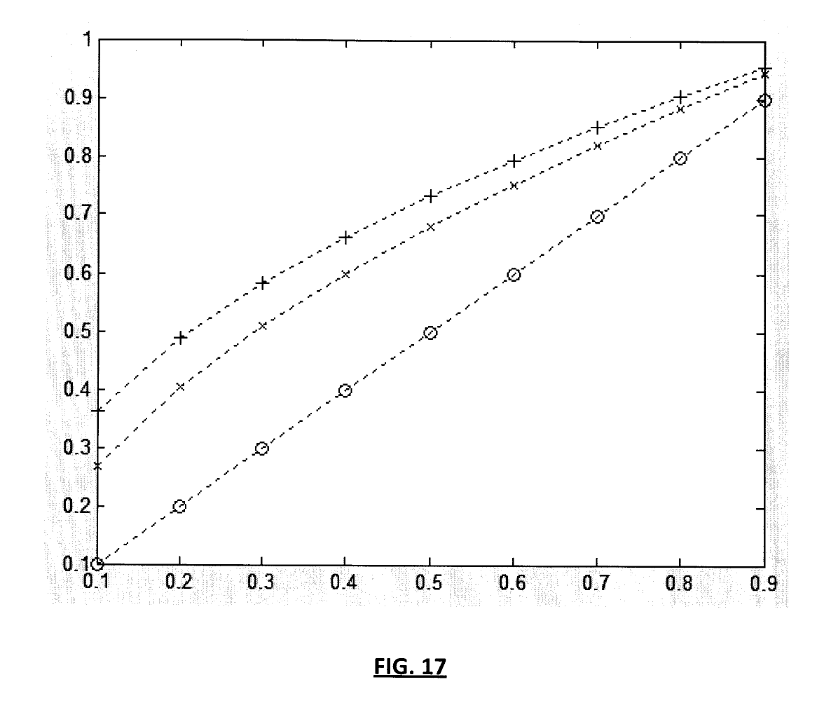

In FIG. 17, an exemplary estimate of coherence and its confidence limits are shown (where confidence limits are low “x”, high “+”, and the estimate of coherence is (o)).

As apparent from FIG. 17, the estimator is biased and there is a relatively small estimation variance. This signifies that the observed variance may be due to patient variability and not computational uncertainty.

The actual coherence that should be used later for classification should be the corrected coherence and not the original estimator. As one can see from FIG. 17, the original estimator has different separation properties than the corrected one. The true coherence is anywhere between the lower and upper bound corresponding to the same abscissa as for the diagonal.

For the sleep markers, corrections were made based on the ontogeny of sleep and measures were employed relative to normal values for age and gender in order to keep the detection within the (−1, 1) hypercube. Details of this process were discussed above.

Three of the different methods of classification were tested, namely the Multilayer Perceptron neural network (MPNN), a probabilistic neural network (PNN) a with a layer of RBF. and the K nearest neighbour (kNN) approach.

Due to the limited number of available patients for these experiments (28 kids and 27 adults), testing methodology included a leave-one-out validation. This procedure takes each patient in sequence and considers it a test example while all the rest of the patients are participating in training the neural network.

Doing this for a set of N patients results in N training sessions and a number of N test cases out of which some will be correctly classified and others not. For each control in the set and each depressed patient, the number of true positive (TP) and number of false positives (FP) was identified. At the end of N training sessions the sensitivity for each class was obtained, control (C) and depressed (D):

$$S ( C ) = \frac{TP ( C )}{TP ( C ) + FN ( C )}$$

$$S ( D ) = \frac{TP ( D )}{TP ( D ) + FN ( D )}$$

In these equations, we have the sensitivity for deciding we have a control when the test case is a control and deciding that the patient is depressed when the test case is actually depressed. These two cases are exhaustive in a binary classification task.

The results for the three methods are:

TABLE 1
Sensitivity adults

| Method | S(C) | S(D) |
|---|---|---|
| kNN | 92 | 83 |
| MLP | 92 | 75 |
| RBF | 92 | 58 |

TABLE 2
Sensitivity kids

| Method | S(C) | S(D) |
|---|---|---|
| kNN | 100 | 75 |
| MLP | 80 | 77 |
| RBF | 30 | 77 |

The interesting observation to note is that when we tested the MLP on kids using microarchitectural parameters only, we obtained consistently S(C)=80% and S(D)=55 while with the extended set of 27 parameters we have obtained S(C)=80% and S(D)=77%.

This shows that microarchitectural elements complement classical sleep markers. As no markers that stand out have been discovered, it appears that the interaction of two or more markers may be significant and highly useful for diagnosing depression.

### Other Applications

In some other applications, the teachings herein may be suitable for use in diagnosing other medical conditions.

For instance, in some cases the teachings herein may have some suitability for predicting of Alzheimer's disease. In particular, the home diagnostic technologies described herein may be useful to monitor patients for sleep abnormalities that are associated with Alzheimer's disease. For example, it has been observed that increased sleep arousal measured for ten days per year or more may be a reasonably good predictor of Alzheimer's disease. Thus, the teachings herein may provide a relatively low cost alternative to imaging diagnostics that are conventionally done for detecting Alzheimer's, which may facilitate the use and prevalence of screening tests.

In some embodiments, the teachings herein may have some suitability for pre-surgical respiratory monitoring.

For instance, the home diagnostic technologies described herein may be suitable for pre-surgical screening of patients in order to predict potential problems that may arise during and after anaesthesia.

It particular, there is a close relationship between sleep and anaesthesia. Clinical studies have shown that patients experiencing respiratory problems during sleep are at risk for developing complications during and after administering various anaesthetic regimens.

There are some indications that pre-surgical screening of respiratory problems during sleep may be quite useful due to significant morbidity and mortality rates associated with problems that arise during and after anaesthesia.

Currently, one prior approach to pre-screening takes into consideration the cerebral aspect of respiration and is possible only through costly tests that available in sleep laboratories (and which may be quite expensive, for example approximately 500$/test). In addition there is a hidden cost to the patient due to travel and possible lost days away from work. Moreover, sleep laboratories may not be able to adequately test the large volume of patients that undergo surgery.

Providing a test in a patient's home according to the teachings herein may thus offer one or more benefits associated with pre-surgical screening. For example, such a solution may not be as limited by the volume of patients. These approaches may also provide a cost reduction per test, in some cases a significant cost reduction. In some cases, the teachings herein may be used to eliminate or at least reduce the costs to the patient for pre-surgery screening. Moreover, by providing for monitoring in a home environment according to the teachings herein, the inconveniences due to travel to the lab and sleeping away from home may be eliminated.

### CONCLUSION

The teachings herein tend to be directed to the difficult task of diagnosing depression by applying a detailed automated characterization of sleep. This includes analyzing sleep continuity, sleep architecture and microarchitecture. This work may be suitable for a method for home implementation, and might be able to open up a new era of diagnosing mental illness with possibilities of remote, unattended tests that may provide one or more benefits over previous diagnostic techniques.

For example, one benefit might include extended diagnostics and screening for sleep laboratories.

Another benefit might be providing at home tests for depression or other medical conditions, and which might be administered by a sleep laboratory or by other personnel, such as a psychiatrist or general practitioner.

In some cases, the teachings herein might be used for pre-surgical respiratory monitoring, which could be managed by an anesthesiologist or other doctor.

In some cases, the teachings herein might be used for predicting Alzheimer's disease.

In some cases, the teachings herein might be used for original equipment manufacturers (OEMs). For instance, the teachings herein could be used to provide a software module (or both) that could be integrated with some other medical apparatus (EEG, CPAP, Holter, etc.)

In some cases, the teachings herein might be used for a combined hardware and software solution. This approach may be particularly useful for general practitioners and psychiatrists, for example, who may not currently have any EEG equipment. Providing a combined hardware and software solution according to the teachings herein may provide a unit that may be easier and more intuitive for general practitioners and psychiatrists to use, as opposed to complicated EEG machines which may be difficult to use and which may require specialized training.

1. A system for diagnosing a medical condition, comprising:
at least one recorder adapted to record brainwaves in a patient and generate sleep data therefrom; and
at least one analyzer block adapted to interpret the sleep data and determine whether the patient is experiencing the medical condition based on a multivariate analysis of at least two biological markers in the sleep data.

2. The system of claim 1, wherein the medical condition is depression.

3. The system of any preceding claim, wherein the biological markers include at least one chronobilogical marker.

4. The system of claim 3, wherein the chronobilogical marker includes an ultradian rhythm for the patient.

5. The system of claim 4, wherein at least one analyzer block is determined to identify at least one of a delay or advance of the ultradian rhythm.

6. The system of claim 4 or claim 5, wherein at least one analyzer block is determined to identify dispersion of the ultradian rhythm of the patient

7. The system of any preceding claim, wherein the biological markers include at least one microarchitectural marker.

8. The system of claim 7, wherein the microarchitectural marker includes at least one of:
(a) the coherence of EEG activity in at least one spectral band;
(b) whole night beta and gamma activity during NREM sleep;
(c) around sleep onset;
(d) REM latency;
(e) REM density; and
(f) SWS time.

9. The system of any preceding claim, wherein the biological markers include at least one macroarchitectural marker.

10. The system of claim 9, wherein the macroarchitectural marker includes at least one of:
(a) altered distribution of slow-wave sleep;
(b) reduced slow-wave sleep;
(c) decreased latency to the first episode of REM sleep;
(d) prolonged first REM period;
(e) increased REM percent; and
(f) increased REM density.

11. The system of any preceding claim, wherein the biological markers include at least one continuity of sleep marker.

12. The system of claim 11, wherein the continuity of sleep marker includes at least one of
(a) sleep latency (SL);
(b) wake after sleep onset (WASO);
(c) number of awakenings (NWAK);
(d) sleep efficiency (SE); and
(e) total sleep time (TST).

13. The system of any preceding claim, wherein the biological markers include at least one estimate of REM density.

14. The system of any preceding claim, wherein the biological markers include at least one coherency analysis.

15. The system of claim 14, wherein the coherency analysis includes a beta bilateral coherency analysis.

16. The system of claim 15, wherein the beta bilateral coherency analysis includes a beta bilateral coherency in at least one hemisphere of the patient's brain

17. The system of claim 14, wherein the coherency analysis includes a theta bilateral coherency analysis.

18. The system of any preceding claim, wherein sleep data is analyzed using a Digital Period Analysis.

19. The system of any preceding claim, wherein sleep data is processed by a diagnostic device.

20. The system of any preceding claim, wherein the sleep data includes raw sleep data.

21. The system of any preceding claim, wherein the sleep data includes processed sleep data.

22. The system of claim 21, wherein the processed sleep data includes a hypnogram.

23. The system of any preceding claim, further comprising an EEG reader adapted to receive EEG data and send the EEG data to a montage block.

24. The system of any preceding claim, wherein at least one recorder is an electroencephalograph.

25. The system of claim 24, wherein the electroencephalograph is adapted for use in a sleep laboratory.

26. The system of claim 24, wherein the electroencephalograph is adapted for use in a home environment.

27. The system of claim 26, wherein the electroencephalograph includes electrodes that are either independent or are part of a net that is adapted to be worn by a patient.

28. The system of claim 23, wherein the montage block sleep data includes a plurality of analyzer blocks.

29. The system of claim 28, wherein the analyzer blocks include at least one chronobilogical, microarchitectural, macroarchitectural, and continuity of sleep blocks.

30. The system of any preceding claim, further comprising a transformer block.

31. The system of claim 30, wherein the transformer block is adapted to compensate for at least one of gender and age.

32. The system of any preceding claim, further comprising a classifier block.

33. The system of claim 32, wherein the classifier block is adapted to perform a classification analysis on the sleep data.

34. The system of claim 33, further comprising a sleep report parser adapted to send prior sleep reports to the analyzer.

35. The system of any one of claims 1 and 3-34, wherein the medical condition is a mood disorder.

36. The system of any one of claims 1 and 3-34, wherein the medical condition is Alzheimer's.

37. The system of any one of claims 1 and 3-34, wherein the medical condition is a respiratory problem.

38. The system of claim 37, wherein the system is operable to detect the respiratory problem as part of a pre-surgical screening.

39. A method of diagnosing a mood disorder according to any one or more of claims 1-34.

40. A system or method for diagnosing a mood disorder including one or more of the elements or steps all as generally and specifically described herein.

41. A system or method for diagnosing a medical condition including one or more of the elements or steps all as generally and specifically described herein.
